(19) United States
(12) Patent Application Publication
(10) Pub. No.: US 2024/0189028 A1
(43) Pub. Date: Jun. 13, 2024

(54) SYSTEMS AND METHODS FOR INCREASING METABOLIC RATES

(71) Applicant: The General Hospital Corporation, Boston, MA (US)

(72) Inventors: Dieter Manstein, Coral Gables, FL (US); Michael Wang-Evers, Charlestown, MA (US); Nunciada Salma, Charlestown, MA (US)

(21) Appl. No.: 18/554,536

(22) PCT Filed: Apr. 11, 2022
(86) PCT No.: PCT/US2022/024315

§ 371 Date: Oct. 9, 2023

Related U.S. Application Data
(60) Provisional application No. 63/173,175, filed on Apr. 9, 2021.

(51) Int. Cl. A61B 18/20; A61B 18/00; A61B 18/12; A61B 18/14

(52) U.S. Cl. CPC A61B 18/203; A61B 18/1206; A61B 18/1477; A61B 2018/00464; A61B 2018/00577; A61B 2018/00702

(57) ABSTRACT

A treatment system can include an energy source configured to thermally damage skin tissue of a subject, a user input device configured to receive a user input, and a computing device that can be configured to receive, from the user input device, the user input indicative of one or more operational parameters of the energy source based on the user input, control the energy source according to the one or more operational parameters to cause the energy source to thermally damage the skin tissue by creating a plurality of treatment regions within a target region of the skin tissue to increase a basal metabolic rate of the subject, wherein the target region includes a non-treatment portion that is interspersed among the treatment regions.

### CROSS-REFERENCE TO RELATED APPLICATIONS

This application claims priority to U.S. Patent Application No. 63/173,175 filed Apr. 9, 2021, and entitled, “Method and Apparatus for Metabolic Enhancement Using Fractional Skin Treatment,” which is hereby incorporated by reference in its entirety.

### STATEMENT REGARDING FEDERALLY SPONSORED RESEARCH

N/A.

### BACKGROUND

The number of individuals that suffer from obesity or are overweight have been continuously increasing. These weight related issues can lead to or can increase the risk of more severe diseases including, for example, high blood pressure, diabetes, heart disease, stroke, sleep apnea, mental illness, pain, and even death. While interventions such as diet and exercise are proven to be effective, these can only go so far. In fact, genetics can play a relatively large role in the outcomes of these interventions. So, even individuals that strictly adhere to these interventions may not achieve their desired fat loss goals.

In some cases, even individuals that are not overweight or obese can still have trouble achieving their fat loss goals. For example, while diet and exercise can decrease the total amount of a fat of an individual, unfortunately, diet and exercise cannot be used to control local fat loss. In other words, diet and exercise cannot target fat loss at specific locations of the body. For example, exercises for a specific muscle group (e.g., bicep curls) will not directly facilitate fat loss at the location of the muscle group (e.g., biceps). Thus, even relatively healthy individuals can still have issues with eliminating undesirable stubborn fat areas.

Thus, it would be desirable to have improved systems and methods for increasing metabolic rates.

### SUMMARY OF THE DISCLOSURE

Some non-limiting examples of the disclosure provide treatment system. The treatment system can include an energy source configured to thermally damage skin tissue of a subject, a user input device configured to receive a user input, and a computing device that can be configured to receive, from the user input device, the user input indicative of one or more operational parameters of the energy source based on the user input, control the energy source according to the one or more operational parameters to cause the energy source to thermally damage the skin tissue by creating a plurality of treatment regions within a target region of the skin tissue to increase a basal metabolic rate of the subject, wherein the target region includes a non-treatment portion that is interspersed among the treatment regions.

In some non-limiting examples, the computing device is further configured to control the energy source according to the one or more operational parameters to randomly distribute the treatment regions among the target region of the skin tissue.

In some non-limiting examples, the creation of the treatment regions are configured to decrease an amount of fat of the subject.

In some non-limiting examples, the plurality of treatment regions form an array of treatment regions within the target region. The array includes multiple columns and multiple rows. The treatment regions are in the multiple columns and the multiple rows of the array.

In some non-limiting examples, the energy source is the transducer that is a light source, and the computing device is further configured to cause the light source to emit light towards the target region of the skin tissue to form the plurality of treatment regions of the skin tissue.

In some non-limiting examples, the computing device is further configured to cause the light source to emit a plurality of beams of light towards the target region of the skin tissue, and each beam of the plurality of beams creates a respective treatment region of the plurality of treatment regions of the skin tissue.

In some non-limiting examples, the light source is configured to generate a fractional illumination pattern that is directed at the target region to form the plurality of treatment regions and the non-treatment region.

In some non-limiting examples, each of the plurality of treatment regions of the skin is a non-ablative treatment region.

In some non-limiting examples, the controller is further configured to control the light source to deliver less than or equal to 9 mJ of energy to create each of the plurality of treatment regions.

In some non-limiting examples, each of the plurality of treatment regions of the skin is an ablative treatment region.

In some non-limiting examples, the controller is further configured to control the light source to deliver less than or equal to 17 mJ of energy to create each of the plurality of treatment regions.

In some non-limiting examples, the target region is at least one of 10 percent of the total body surface area of the skin tissue of the subject, 20 percent of the total body surface area of the skin tissue of the subject, 30 percent of the total body surface area of the skin tissue of the subject, or 32 percent of the total body surface area of the skin tissue of the subject.

In some non-limiting examples, the target region does not include at least one of the genitals of the subject, or the head of the subject.

In some non-limiting examples, each of the plurality of treatment regions defines a treatment surface within the target region, and the non-treatment region defines a non-treatment surface within the target region. All the treatment surfaces of the plurality of treatment regions defines a total treatment surface area of the target region. The percentage of the treatment surface area to total surface area of the target region is at greater than or equal to 10 percent.

In some non-limiting examples, the percentage of the treatment surface area to the total surface area of the treatment region is at least one of greater than or equal to 15 percent, 20 percent, 30 percent, or 32 percent.

In some non-limiting examples, each of the plurality of treatment regions defines a treatment surface, and the non-treatment region defines a non-treatment surface, all the treatment surfaces of the plurality of treatment regions defines a total treatment surface area of the target region, and the percentage of the treatment surface area to the total body surface of the subject is at least 1 percent.

In some non-limiting examples, the percentage of the treatment surface area to the total body surface of the subject is at least one of 2 percent, 3.6 percent, or 6.3 percent.

In some non-limiting examples, the formation of the plurality of treatment regions of the target region of the skin tissue decreases an amount of fat tissue at the target region.

In some non-limiting examples, the formation of the plurality of treatment regions of the target region of the skin tissue decreases a total amount of fat of the subject.

In some non-limiting examples, the formation of the plurality of treatment regions of the target region of the skin tissue transforms a fat cell that is white fat cell or beige fat cell into a brown fat cell.

In some non-limiting examples, the formation of the plurality of treatment regions of the target region of the skin tissue increases the amount of noradrenaline circulating through the bloodstream of the subject.

In some non-limiting examples, the energy source includes a transducer.

In some non-limiting examples, the energy source includes an electrical generator and one or more electrodes, the electrical generator being configured to direct an electrical signal to the one or more electrodes thereby thermally damaging the skin tissue.

In some non-limiting examples, the energy source is configured to create the plurality of treatment regions without creating an incision or a puncture at the target region of the skin tissue.

In some non-limiting examples, a treatment region of the plurality of treatment regions has a width of less than or equal to 1 millimeter.

In some non-limiting examples, a treatment region of the plurality of treatment regions does not extend into the subcutaneous tissue of the treatment region.

Some embodiments of the discourse provide a treatment system that can include an energy source configured to thermally damage skin tissue of a subject, a user input device configured to receive a user input, and a computing device that can be configured to receive, from the user input device, the user input indicative of one or more operational parameters of the energy source, and based on the user input, control the energy source according to the one or more operational parameters to cause the energy source to thermally damage the skin tissue by creating a plurality of treatment regions within a target region of the skin tissue to decrease a total amount of fat of the subject, wherein the target region includes a non-treatment portion that is interspersed among the treatment regions.

Some embodiments of the discourse provide a treatment system that can include an energy source configured to thermally damage skin tissue of a subject, a user input device configured to receive a user input, and a computing device that can be configured to receive, from the user input device, the user input indicative of one or more operational parameters of the energy source, and based on the user input, control the energy source according to the one or more operational parameters to cause the energy source to thermally damage the skin tissue by creating a plurality of treatment regions within a target region of the skin tissue to transform one or more white fat cells into one or more beige fat cells or one or more brown fat cells, wherein the target region includes a non-treatment portion that is interspersed among the treatment regions.

Some embodiments of the discourse provide a treatment system that can include an energy source configured to thermally damage skin tissue of a subject, a user input device configured to receive a user input, and a computing device that can be configured to receive, from the user input device, the user input indicative of one or more operational parameters of the energy source, and based on the user input, control the energy source according to the one or more operational parameters to cause the energy source to thermally damage the skin tissue by creating a plurality of treatment regions within a target region of the skin tissue to increase the amount of noradrenaline circulating through the bloodstream of the subject, wherein the target region includes a non-treatment portion that is interspersed among the treatment regions.

Some embodiments describe a method of increasing a metabolic rate. The method can include directing energy, using an energy source, at a target region in skin tissue of a subject, creating a plurality of treatment regions in the target region of the skin tissue, from the energy interacting with the skin tissue at the target region, the plurality of treatment portions being interspersed among an untreated region of the target region of the skin tissue, and increasing the basal metabolic rate of the subject, from the creation of the plurality of treatment regions.

In some non-limiting examples, each treatment portion has a width that is less than or equal to 1 millimeter.

In some non-limiting examples, the method can include at least one of decreasing an amount of fat at the target region of the skin tissue, from the creation of the plurality of treatment regions, decreasing an amount of fat at a region of the skin tissue that is different than the target region, from the creation of the plurality of treatment regions, decreasing a thickness of fat at the target region of the skin tissue, from the creation of the plurality of treatment regions, decreasing a thickness of fat at the region of the skin tissue that is different than the target region, from the creation of the plurality of treatment regions, or decreasing a total amount of fat of the subject, from the creation of the plurality of treatment regions.

In some non-limiting examples, the method can include converting at least one white fat cell into a beige or a brown fat cell, from the creation of the plurality of treatment regions.

In some non-limiting examples, the method can include increasing the concentration of at least one hormone circulating in the subject, from the creation of the plurality of treatment regions, or increasing the concentration of at least one neurotransmitter circulating in the subject, from the creation of the plurality of treatment regions.

In some non-limiting examples, the at least one hormone or the at least one neurotransmitter is norepinephrine.

In some non-limiting examples, the plurality of treatment regions are created without puncturing or incising the skin tissue.

In some non-limiting examples, the method can include moving the energy source to the target region, and with the energy source stationary, directing energy at the target region from the energy source to create the plurality of treatment regions.

In some non-limiting examples, the method can include with the energy source stationary, directing first energy at the target region from the energy source to create a first subset of the plurality of treatment regions, and with the energy source stationary, directing second energy at the target region from the energy source to create a second subset of the plurality of treatment regions.

In some non-limiting examples, the first subset of treatment regions is a first row of treatment regions, and the second subset of the treatment regions is a second row of treatment regions.

In some non-limiting examples, the method can include the first subset of treatment regions is a first column of treatment regions, and the second subset of the treatment regions is a second column of treatment regions.

In some non-limiting examples, the method can include the target region is a first target region and the method can include moving the energy source to second target region that is different than the first target region, and with the energy source stationary, directing energy at the second target region from the energy source to create another plurality of treatment regions within the second target region, the another plurality of treatment regions being interspersed with a plurality of non-treatment regions within the second target region of the skin tissue.

Some embodiments of the disclosure provide a method of improving a weight disorder. The method can include directing energy, using an energy source, at a target region in skin tissue of a subject, creating a plurality of treatment regions in the target region of the skin tissue, from the energy interacting with the skin tissue at the target region, the plurality of treatment regions being interspersed among an untreated region of the target region of the skin tissue, increasing the basal metabolic rate the subject, from the creation of the plurality of treatment regions, decreasing an amount of fat of the subject, based on the increasing the basal metabolic rate of the subject, and improving the weight disorder from the decreasing of the amount of fat of the subject.

In some non-limiting examples, each treatment region has a width that is less than or equal to 1 millimeter.

In some non-limiting examples, the method can include at least one of decreasing the amount of fat at the target region of the skin tissue, from the creation of the plurality of treatment regions, decreasing the amount of fat at a region of the skin tissue that is different than the target region, from the creation of the plurality of treatment regions, decreasing a thickness of fat at the target region of the skin tissue, from the creation of the plurality of treatment regions, decreasing a thickness of fat at the region of the skin tissue that is different than the target region, from the creation of the plurality of treatment regions, or decreasing a total amount of fat of the subject, from the creation of the plurality of treatment regions.

In some non-limiting examples, the method can include converting at least one white fat cell into a beige or a brown fat cell, from the creation of the plurality of treatment regions.

In some non-limiting examples, the method can include increasing the concentration of at least one hormone circulating in the subject, from the creation of the plurality of treatment regions, or increasing the concentration of at least one neurotransmitter circulating in the subject, from the creation of the plurality of treatment regions.

In some non-limiting examples, the at least one hormone or the at least one neurotransmitter is norepinephrine.

In some non-limiting examples, the method can include the plurality of treatment regions are created without puncturing or incising the skin tissue.

In some non-limiting examples, the method can include improving one or more diseases caused by the weight disorder, from the creation of the plurality of treatment regions.

In some non-limiting examples, the one or more diseases include at least one of diabetes, heart disease, high blood pressure, mental illness, pain, high cholesterol, or high triglyceride levels.

Some embodiments provide a method of improving one or more diseases. The method can include directing energy, using an energy source, at a target region in skin tissue of a subject, creating a plurality of treatment regions in the target region of the skin tissue, from the energy interacting with the skin tissue at the target region, the plurality of treatment regions being interspersed among a non-treatment region of the target region of the skin tissue, decreasing an amount of fat of the subject, based on the creation of the plurality of treatment regions, and improving the one or more diseases, based on the decreasing the amount of fat of the subject.

In some non-limiting examples, the one or more diseases include at least one of diabetes, heart disease, high blood pressure, mental illness, pain, high cholesterol, or high triglyceride levels.

The foregoing and other aspects and advantages of the present disclosure will appear from the following description. In the description, reference is made to the accompanying drawings that form a part hereof, and in which there is shown by way of illustration one or more exemplary versions. These versions do not necessarily represent the full scope of the disclosure.

### BRIEF DESCRIPTION OF THE DRAWINGS

The following drawings are provided to help illustrate various features of non-limiting examples of the disclosure, and are not intended to limit the scope of the disclosure or exclude alternative implementations.

FIG. 1 shows a schematic illustration of a treatment system.

FIG. 2A shows a schematic top view of a target region of skin tissue of a subject, which includes a plurality of treatment regions, and a non-treatment region.

FIG. 2B shows a cross-section of the target region of FIG. 2A, taken along line 2B-2B of FIG. 2A.

FIG. 2C shows an example of a subject having multiple target regions that have been treated.

FIG. 2D shows an example of a subject having a large target region that has been treated.

FIG. 2E shows an example of a subject having another large target region that has been treated.

FIG. 3A shows a schematic illustration of another treatment system.

FIG. 3B shows a cross-sectional view of the treatment system of FIG. 3A with respect to a target region of skin tissue.

FIG. 3C shows an illustration of a shield that can include a slot, and an actuator coupled to the shield.

FIG. 4 shows a schematic illustration of another treatment system.

FIG. 5 shows a schematic illustration of another treatment system.

FIG. 6 shows a schematic illustration of another treatment system.

FIG. 7 shows a schematic illustration of another treatment system.

FIG. 8 shows a schematic illustration of another treatment system.

FIG. 9 shows a schematic illustration of another treatment system.

FIG. 10A shows a side view of a schematic illustration of another treatment system.

FIG. 10B shows a front view schematic illustration of the treatment system of FIG. 10A.

FIG. 11A shows a front schematic view of an alternative configuration of the slide of the treatment system of FIG. 10A.

FIG. 12 shows a side view of a schematic illustration of another treatment system.

FIG. 13 shows a side view of a schematic illustration of another treatment system.

FIG. 14 shows a side view of a schematic illustration of another treatment system.

FIG. 15 shows a schematic illustration of the Rule of Nines.

FIG. 16 shows a flowchart of a process of at least one of increasing a metabolism of a subject, improving a weight disorder of the subject, improving one or more diseases associated with the weight disorder, decreasing a total amount of fat of the subject, decreasing a total weight of a subject, etc.

FIG. 17 shows a graphical representation to illustrate the concept of confluent laser treatments and fractional laser treatments.

FIG. 18 shows a photograph of a mouse of the experimental setup.

FIG. 19 shows a photograph of a non-ablation FP (“nFP”) on one leg of a subject with 35 mJ per treatment region and a density of 11% (of treatment regions), and an ablation FP (“aFP”) on the other leg of the subject with 20 mJ per treatment region and a density of 15% (of treatment regions).

FIG. 20 shows a positron emission tomography (“PET”) image of both legs of the subject of FIG. 19.

FIG. 21 shows a graph of body mass versus body surface area (“BSA”) versus body mass with a fitted function (e.g., using the Meeh equation).

FIG. 22 shows a graph of total energy expenditure for six groups, and a graph of total water consumption for the six groups.

FIG. 23 shows a graph of total energy expenditure for six groups, and a graph of total water consumption for the six groups.

FIG. 24 shows a graph of the average daily energy expenditure for six groups before and after treatment.

FIG. 25 shows a graph of the energy expenditure over time for six groups.

FIG. 26 shows a graph of the energy expenditure over time for six groups.

FIG. 27 shows a bar graph of the total energy expenditure for six groups.

FIG. 28 shows a bar graph of the total energy expenditure for six groups.

FIG. 29 shows a bar graph of the average daily energy expenditure for a first set of six groups, and a second set of six groups.

FIG. 30 shows a graph of the energy expenditure over time for six groups.

FIG. 31 shows a graph of the energy expenditure over time for six groups.

FIG. 32 shows a bar graph of the total energy expenditure for ten groups.

FIG. 33 shows a bar graph of the total energy expenditure for ten groups.

FIG. 34 shows a bar graph of the fat loss using EchoMRI for seven groups, and a bar graph of the weight loss using EchoMRI for the seven groups.

FIG. 35 shows a bar graph of the fat loss using EchoMRI for seven groups, and a bar graph of the weight loss using EchoMRI for the seven groups.

FIG. 36 shows photographs of mice from the ablative FP group.

FIG. 37 shows photographs of mice from the non-ablative FP group.

FIG. 38 shows images of white adipose tissue for different treatment groups.

FIG. 39 shows a graph of the noradrenaline concentration for the ablative laser groups, and a graph of the noradrenaline concentration for the non-ablative laser group.

FIG. 40 shows a graph of the IL-6 concentration for the ablative laser groups, and a graph of the IL-6 concentration for the non-ablative laser group.

### DETAILED DESCRIPTION OF THE PRESENT DISCLOSURE

As described above, individuals can have trouble eliminating total fat loss and targeting fat loss to specific local areas, both of which can be even more difficult during the aging process (e.g., because baseline metabolic rate decreases as individuals age). While diet and exercise can help with total fat loss, diet and exercise cannot locally target and eliminate fat. Correspondingly, conventional procedures including liposuction can locally target fat, but cannot eliminate fat outside of the treatment area. Interestingly, victims of severe burns can exhibit pathophysiological stress responses that can include a hypermetabolic response for extended times, which can unfortunately last for months or longer. These burn-induced metabolic responses can be more pronounced with greater degrees of trauma, and can lead to significant weight loss and certain health complications, such as cachexia, reduced immune function, liver problems (e.g., hepatic steatosis), sepsis, multiple organ dysfunction syndrome, etc.

Accordingly, there can be a need for systems and devices that can be configured to produce well-tolerated damage (e.g., fractional damage) over large regions of skin tissue to enhance metabolism and potentially produce beneficial effects such as, for example, desirable weight loss including fat loss, ameliorate a metabolic syndrome, improve insulin resistance, etc., while avoiding the severe trauma, immune system impairment, and other severe issues that may result from severe burns.

Some non-limiting examples of the disclosure provide advantages to these issues (and others) by providing improved systems and methods for increasing metabolic rates. For example, some non-limiting examples of the disclosure provide a treatment system that can include an energy source (e.g., a laser) and a computing device configured to control the energy source according to one or more operational parameters (e.g., pulse duration, pulse width, total energy delivered, total duration of energy for a target region of the skin tissue, etc.). When the computing device controls the energy source according to the one or more operational parameters, the skin tissue at a target region can be thermally damaged in a controlled manner. In this way, the treatment system can not only advantageously increase the metabolic rate at the target region of the skin tissue that receives the energy and increase the basal metabolic rate of the subject, but the treatment system can also thermally damage the skin in a safe manner that facilitates quick healing with minimal side effects. For example, the skin tissue can be thermally damaged according to a fractional pattern with a plurality of treatment regions (e.g., that receive the energy from the energy source) and non-treatment region (e.g., that does not receive the energy from the energy source) interspersed with the plurality of treatment regions (and vice versa). Thus, in some cases, with the interspersion of non-treated regions (e.g., healthy tissue) with treated regions (e.g., thermally damaged tissue), and the relatively small size of the treated regions (e.g., less than 1 millimeter in width), the treated regions can heal quickly. Accordingly, this can facilitate an increase in metabolic rates (e.g., which can lead to certain beneficial responses) in a controlled manner (e.g., if the trauma to the body is not too severe). For example, unlike uncontrolled severe burns that could undesirably increase the metabolic rate for months or longer and that may not heal properly (e.g., with scars), the controlled thermal damage provided by the treatment system can quickly and entirely heal the thermally damaged regions without lasting damage, and the metabolic rate can be advantageously increased for a much shorter duration (e.g., 1 week).

In some non-limiting examples, the treatment system can implement a fractional skin treatment (also known as fractional resurfacing) on skin tissue. Fractional skin treatment is a cosmetic procedure that includes the formation of small regions of damage in skin tissue (e.g., ablation or thermal damage) that are surrounded by healthy tissue. Fractional treatments can be well-tolerated by the body because of the small size of the damaged regions (e.g., generally less than about 1 mm) and proximity of healthy tissue. The locally dispersed (or “fractional”) nature of such thermal damage can facilitate a rapid healing of the damaged regions, as well as other desirable effects such as tissue shrinkage. Fractional resurfacing can be performed on the facial region, although other body areas can also be treated fractionally. Procedures and devices for generating this fractional damage in biological tissue have been gaining increased attention and usage. The discontinuous small regions of damage can be produced using certain types of lasers or other energy-based devices that can interact with skin tissue to generate small regions of ablated or thermally-damaged tissue. This fractional damage can be well-tolerated, and in some cases, cosmetic patients may feel a sensation comparable to a slight sunburn in the treated area after a procedure.

Some non-limiting examples of this disclosure provide safe methods and devices for increasing body metabolism by generating fractional thermal damage to skin tissue over a significant region of the body (e.g., greater than 10 percent of the total body surface area of the subject). Such fractional damage can be well-tolerated, and the increased metabolic rate can lead to desirable weight loss and other beneficial health effects without the need for strenuous exercise or diet regimens.

In non-limiting examples of the disclosure, fractional damage can be produced over a percentage of the skin surface that is greater than that for conventional cosmetic treatments. Typical cosmetic fractional treatments involve only the facial region, hands, or parts of the chest, treating less than about 5% of the total skin surface area. In contrast, non-limiting examples of the present disclosure include generating fractional damage over at least about 20% of the skin's surface area. The extended amount of fractional damage can be significant enough to generate an overall increase in metabolic rate while still being well-tolerated and avoiding undesirable health issues that can face victims of severe burn trauma (e.g., the thermally damaged regions of the skin can quickly heal with little to no lasting damage). As with cosmetic fractional treatments, the subject can experience mild discomfort that is comparable to a mild sunburn over the treated area, or other effects such as some scabbing or oozing that heals over time.

In some non-limiting examples, the fractional damage can be ablative, where small regions of tissue (e.g., less than about 1 mm in width, less than 1 mm in diameter, etc.) extend (e.g., are vaporized) to a depth within the dermis. In some cases, laser and optical systems can be provided that can produce such ablative fractional damage (e.g., for cosmetic purposes such as skin tightening). These laser and optical systems, together with appropriate parameters (e.g., energy parameters), can generate desired amounts of thermal ablation configured to elicit increases in metabolism of the subject, decreases in fat of the subject, treatment of one or more diseases associated with weight disorders (e.g., obesity, being overweight, etc.) including diabetes, high blood pressure, heart disease, mental illness, pain, etc., by decreasing the fat of the subject, etc. In non-limiting examples of the disclosure, the local fraction of irradiated skin tissue surface in a treated region can be between about 10% and 30%, with the other 70%-90% of skin surface surrounding the ablated spots remaining largely undamaged.

In some non-limiting examples, the fractional damage can be generated non-ablatively, for example, such that thermally-damaged regions are generated in the skin tissue but no tissue is vaporized. In some cases, the width of such small regions of thermal damage can be, for example, less than about 1 mm in width, less than about 0.5 mm in width, etc., with the thermally-damaged regions extending to a depth within the dermis. In some cases, the lasers and optical systems presented herein that can produce such non-ablative fractional damage can be similar to those used in cosmetic procedures. In non-limiting examples of the disclosure, the fraction of non-ablative damaged skin surface can be between about 10% and 30%, with the other 70%-90% of skin surface surrounding the ablated spots remaining largely undamaged.

In some non-limiting examples, a computing device can control the treatment system according to one or more parameters that can create the desired thermal damage in the skin tissue to elicit the desired effects presented herein. In some cases, the one or more parameters can include a laser wavelength (e.g., when the energy source is a laser), energy of the energy source, intensity of the energy source, fluence delivered by the energy source, beam width of the energy source, duration of each pulse or the total amount of energy delivered by the energy source to a target region of the skin tissue during a period of time, combinations thereof, etc. In some cases, the one or more parameters can correspond to parameters used for analogous cosmetic procedures. In some preferable cases, the one or more parameters can be comparable to those for more “aggressive” cosmetic treatments, such that a greater degree of local thermal damage can be generated to elicit the desired response, while still being well-tolerated.

In non-limiting examples of the disclosure, fractional damage can be generated over a large region of skin. For example, such damage can be produced over a major region of the back and optionally buttocks, over the chest and abdominal area, over a large region of the surface of one or more limbs (e.g., a leg, an arm, etc.), etc. The total area covered by fractional damage in a single treatment can be greater than about 20% of the total body surface, greater than about 30% of the total body surface, etc.

In some non-limiting examples, subsequent fractional treatments can be applied after a relatively short time interval, for example, of substantially (i.e., deviating by less than 10 percent from) 1-2 weeks. Such subsequent treatments can be applied to one or more regions of the body that are different than that of the prior treatment. In this manner, fractional damage to the skin over a significant amount of the body can be achieved within a relatively short timeframe while avoiding multiple treatments to the same region. In certain non-limiting examples, three or more such treatments can be provided at relatively short intervals, preferably in different regions of the body.

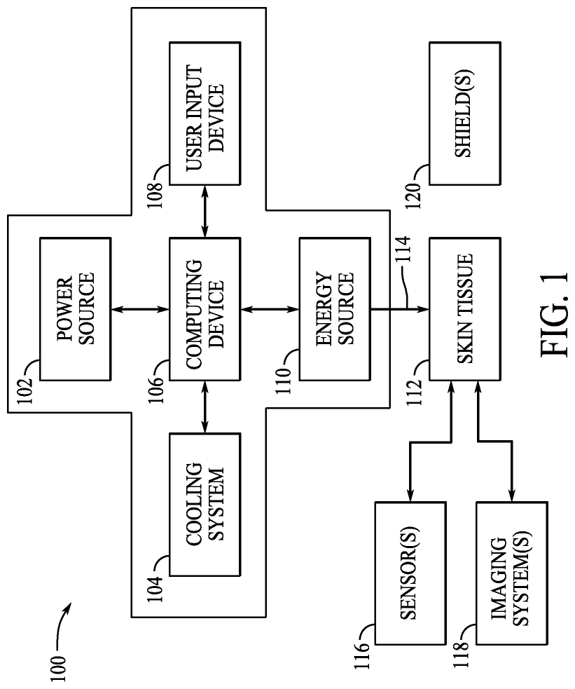

FIG. 1 shows a schematic illustration of a treatment system 100. The treatment system 100 can include a power source 102, a cooling system 104, a computing device 106, a user input device 108, and an energy source 110. The power source 102 can be implemented in different ways, and can provide power (e.g. electrical power) to some or all of the components of the treatment system 100. For example, the power source 102 can provide power to the cooling system 104, the computing device 106, the user input device 108, the energy source 110, etc. In some cases, the power source 102 can be an electrical power source, such as, for example, an electrical storage device (e.g., one or more batteries, a capacitor such as a super capacitor, a rechargeable battery (e.g., a lithium-ion battery)), a power supply, an electrical power cord (e.g., that receives power from an electrical outlet), etc.

The cooling system 104 can cool the skin tissue of the subject before, during, or after the application of energy to the skin tissue by the energy source 110. In some cases, the cooling system 104 can be an evaporative cooling system, which can circulate heat transferring fluid (e.g., a refrigerant) that can absorb heat from the skin tissue, and transmit the heated fluid to an evaporator that can include a fan to remove heat from the fluid. In other cases, the cooling system 104 can include a fan that can blow air across the skin tissue (e.g., direct air at the skin tissue) thereby cooling the skin tissue.

In some non-limiting examples, the computing device 106 can be in communication (e.g., bidirectional communication) with some or all of the components of the treatment system 100. For example, the computing device 106 can be in communication with the power source 102, the cooling system 104, the user input device 108, the energy source 110, etc., to transmit instructions to (or receive data from) a respective component of the treatment system 100. In some cases, this can include the computing device 106 controlling the energy source 110 to deliver energy to the skin tissue of the subject according to one or more parameters to elicit a desired response in the skin tissue (and other regions of the body more generally). The computing device 106 can be implemented in a variety of ways. For example, the computing device 106 can be implemented as one or more processor devices of known types (e.g., microcontrollers, field-programmable gate arrays, programmable logic controllers, logic gates, etc.), including as general or special purpose computers. In addition, the computing device 106 can also include other computing components, such as memory, inputs, other output devices, etc. (not shown). In this regard, the computing device 106 can be configured to implement some or all of the steps of the processes described herein, as appropriate, which can be retrieved from memory. In some non-limiting examples, the computing device 106 can include multiple control devices (or modules) that can be integrated into a single component or arranged as multiple separate components.

In some non-limiting examples, the user input device 108 can be configured to receive one or more user inputs from a user, which can be received by the computing device 106 and can be used to control the energy source 110. For example, the computing device 106 can receive, from the user input device 108, a user input indicative of the one or more operational parameters of the energy source, and can control the energy source 110 according to the one or more operational parameters to thermally damage the skin tissue to elicit the desired response. In this way, a user can control the operation of the energy source to elicit the desired response. In some cases, the user input device 108 can facilitate receiving (or otherwise determining) the one or more parameters of the energy source. For example, a user input device can receive a user input indicative of the one or more parameters of the energy source. In this way, a user can manually adjust the one or more parameters for a specific subject. For example, each subject can have different skin tones, skin thicknesses, total body surfaces, total body weight, etc., which can impact the one or more laser parameters. Accordingly, a computing device can determine the one or more parameters of the energy source, based on the user input, which can be indicative of the presence (or absence) of a particular skin tone, the presence (or absence) of a particular skin thickness, the total body surface of the subject, the total body weight of the subject, etc.

The user input device 108 can be implemented in different ways. For example, the user input device 108 can include a button, a switch, a lever, a slider, a touchscreen, a mouse, a keyboard, a microphone, etc. In some cases, actuation of a user input device can generate signals in the form of electrical signals, which can be received by the computing device 106 and utilized accordingly. In some non-limiting examples, the user input device 108 can include a user interface.

In some non-limiting examples, the energy source 110 can be configured to thermally damage the skin tissue 112 of a subject (e.g., in a fractional pattern), which can increase a metabolic rate of a target region of the skin tissue 112 or a basal metabolic rate of the subject (e.g., and thus increase a metabolic rate of a different region of the skin tissue 112 other than the target region). For example, the energy source 110 can be configured to deliver energy 114 to the skin tissue 112 to create a plurality of treatment regions (e.g., each treatment region being thermally damaged) in a target region of the skin tissue 112 that are separated by at least one non-treatment region of the skin tissue 112 (e.g., each non-treatment region not being thermally damaged). In some cases, the non-treatment region of the target region of the skin tissue 112 can be interspersed among the plurality of treatment regions in the target region of the skin tissue 112. For example, the non-treatment region can be contiguous and the plurality of treatment regions can surround the non-treatment region. In some configurations, two treatment regions can be separated by greater than 1 millimeter, and in some cases, each treatment region can be separated by an adjacent treatment region by greater than or equal to 1 millimeter. In this way, with sufficient spans of the non-treatment region between the treatment regions can allow for better and quicker healing (e.g., nutrient diffusion into the treatment regions).

In some non-limiting examples, the plurality of treatment regions of the target region of the skin tissue 112 can be an array, or can be in a random pattern. For example, the array can include multiple rows and multiple columns, and the plurality of treatment regions can be in the multiple rows and the plurality of treatment regions can be in the multiple columns. In particular, at least two treatment regions can be aligned with each other and in a first row of the array, and at least two other treatment regions can be aligned with each other and in a second row of the array different from the first row of the array. Correspondingly, at least two treatment regions can be aligned with each other and in a first column of the array, and at least two other treatment regions can be aligned with each other and in a second column of the array different from the first column of the array. In other configurations, the treatment regions can be randomly distributed throughout the target region (e.g., created from a fractional laser pattern). In some configurations, each treatment region can have a width (e.g., a diameter) that is less than or equal to 1 millimeter, less than or equal to 0.75 millimeters, less than or equal to 0.5 millimeters, less than or equal to 0.25 millimeters, etc. In this way, with relatively small widths of treatment regions (e.g., substantially 1 millimeter in diameter), the treatment regions are more likely to heal faster and with less lasting damage (e.g., scarring). In some cases, a treatment region can be a microscopic treatment zone of target region of the skin tissue 112. In some configurations, a treatment region can be defined as a thermal damage zone, a dermal damage zone, etc. In some configurations, a treatment region can be defined as a thermal treatment zone.

In some non-limiting examples, the energy source 110 can deliver energy to multiple target regions of the skin tissue 112 that can span a substantial region of the entire body surface area of the skin tissue of the subject (e.g., unlike other fractional configurations), and the target region of the skin tissue 112 can span a substantially larger region of a skin tissue surface area, as compared to previous fractional lasers. For example, the target region of the skin tissue that receives the energy 114 (e.g., a single pulse of energy, such as laser energy, a single can of the laser, a single application of the fractional laser pattern, etc.) can be larger than 10 cm², which is different than conventional approaches of fractional therapy. For example, the target region of skin tissue for facial resurfacing procedures is typically much lower than 10 cm² (e.g., due to the curvature of the face, the need for tight control of the laser because the laser is close to delicate anatomical structures including the eyes, etc.). Conversely, because thermal treatment of the skin tissue 112 as described herein includes much larger swaths of the skin tissue 112 (e.g., which may be required to elicit the systemic metabolic response), and does need to be directed at specific anatomical structures (e.g., to elicit the response at a desired target does not require thermal treatment of that target), the thermal treatment can be directed over far less sensitive structures (e.g., the back of the subject, the stomach of the subject, etc.), and the target region can be made to be much larger. Thus, the target region can be larger than 10 cm², larger than 20 cm², larger than 30 cm², larger than 40 cm², larger than 50 cm², etc. In some configurations, the target region of the skin tissue 112 is the region of the skin tissue 112 that can receive the energy 114 without moving the treatment system 100 (e.g., the energy source 110, which can include a component to deliver the energy 114, such as an optical fiber).

In some non-limiting examples, each of the plurality of treatment regions can be ablative, can be non-ablative, or can be a combination of ablative and non-ablative. For example, each treatment region can be ablative, in which the tissue can be at least partially vaporized at the location, or each treatment region can be non-ablative, in which the tissue is not vaporized at the location. In some configurations, each treatment region that is ablative (e.g., an ablative treatment zone) can create a corresponding hole in the tissue. For example, each treatment region that is ablative can form a hole in the skin tissue (e.g., a blind hole), in which the skin tissue at the treatment region vaporizes. In some cases, the one or more treatment parameters (or features of the energy source 110, such as the operating wavelength of the energy source) can determine whether the treatment regions are ablative or non-ablative when the energy 114 is delivered to the skin tissue 112. For example, if the wavelength of the laser coincides more with the absorption coefficient of water, then the water in the tissue absorbs greater amounts of energy from the laser leading to ablative tissue damage.

In some non-limiting examples, the target region (or multiple target regions together) can span relatively large regions of non-sensitive regions of the body to elicit the desired response (e.g., increase the basal metabolic rate of the subject). For example, a single target region, which can be defined by a boundary of treatment regions of the skin tissue 112 at the respective target area (e.g., the plurality of treatment regions forming a perimeter that defines the respective target region), or multiple target regions can collectively cover greater than 5 percent of the total body surface area of the subject (e.g., in which conventional fractional treatment can cover significantly less than 5 percent of the total body surface area (“BSA”) of the subject). In some cases, the target region (or multiple target regions) can cover at least 10 percent of the total BSA of the subject, 20 percent of the total BSA of the subject, 30 percent of the total BSA of the subject, 32 percent of the total BSA of the subject. In some non-limiting examples, a first target region can be separated from a second target region.

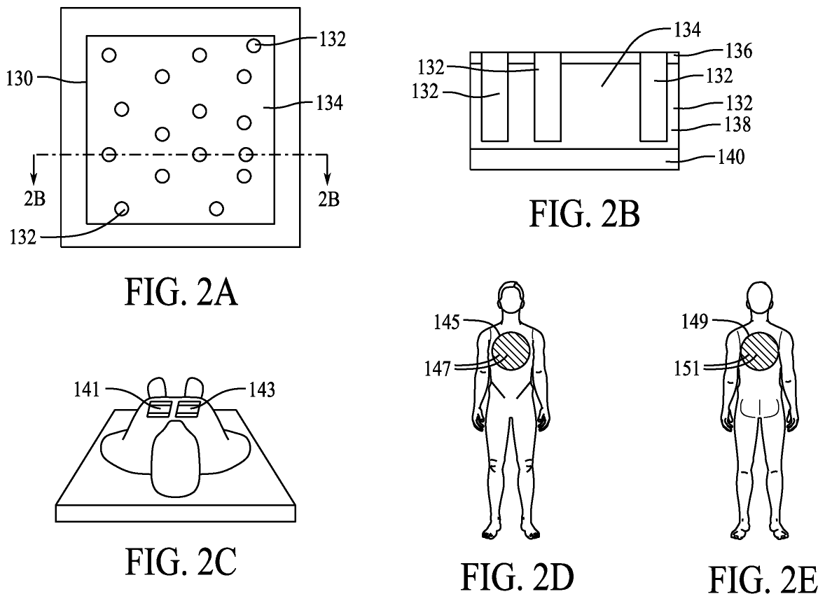

FIG. 2A shows a schematic top view of a target region 130 of skin tissue of a subject, which includes a plurality of treatment regions 132 (e.g., that are thermally damaged, denoted by a circle in FIG. 2A), and a non-treatment region 134 (e.g., that is not thermally damaged, denoted by the regions between the plurality of treatment regions 132) The target region 130 of skin tissue is an example of the thermal damage pattern (e.g., in a fractional pattern) that occurs when the energy 114 is delivered to the target region of the skin tissue 112. As shown in FIG. 2A, the plurality of treatment regions 132 are separated by the non-treatment region 134 (e.g., the treatment regions 134 are interspersed among the non-treatment region 134). In other words, the non-treatment region 134 of the target region 130 extends between the plurality of treatment regions 132, in which the non-treatment region 134 can be contiguous. In some non-limiting examples, while the treatment regions 132 are illustrated as being randomly distributed throughout the treatment region 130, in other configurations, the treatment regions 132 can be in an array. In some cases, a non-treatment region 134 can be referred to as an untreated region.

In some non-limiting examples, the treatment region 130 can be defined by the treatment regions 132. For example, treatment regions 132 at opposing ends can determine a dimension (e.g., a width, a length, a diagonal, a perimeter, etc.) of the treatment region 130. For example, a subset of the plurality of treatment regions 132 at the periphery of the target region 130 can define the boundary of the target region 130 (e.g., the area enclosed by and including the peripheral treatment regions 132 define the target region 130).

In some cases, the size of each treatment region 132 can be substantially the same as each other (e.g., a width, a cross-sectional area, a depth, a top surface area, etc.), and the non-treatment region 132 can be (substantially) larger than a treatment region 132. While each of the treatment regions 132 are illustrated as being circle in cross-section, in other configurations, the treatment regions 132 can have other cross-sectional shapes (e.g., oval, etc.). In some cases, the one or more parameters of the energy source 110 can dictate the particular cross-sectional shape of a treatment region. For example, when the energy source 110 is laser, the one or more parameters can include the Rayleigh length of the laser, which can dictate how much the laser beam diverges, which can thus dictate the width of the laser beam (e.g., when the laser beam interacts with the skin tissue) and thus the width of the treatment region.

In some non-limiting examples, the one or more parameters of the energy source 110 can determine the density of the treatment regions 132 relative to the non-treatment region 134, or the total surface area of the target region 130 of the skin tissue. For example, each treatment region 132 can have a treatment surface (e.g., the entire top surface of the treatment region that is thermally damaged), and each non-treatment region 132 can have a non-treatment surface (e.g., the entire top surface of the non-treatment region 134 that is not thermally damaged). In some cases, all the treatment surfaces of the plurality of treatment regions 132 can define a total treatment surface area of the target region 130, and the non-treatment surface of the non-treatment region 134 can define a total non-treatment surface area of the target region 130. In some cases, the percentage of the total treatment surface area of the target region 130 to the total surface area of the target region 130 can be greater than or equal to 10 percent, greater than or equal to 15 percent, greater than or equal to 20 percent, greater than or equal to 30 percent, greater than or equal to 32 percent, etc. Correspondingly, in some cases, the total non-treatment surface area of the target region 130 to the total surface area of the target region 130 can be less than or equal to 68 percent, less than or equal to 70 percent, less than or equal to 80 percent, less than or equal to 90 percent, etc.

In some non-limiting examples, the one or more parameters of the energy source 110 can determine the absolute treated surface area of the subject. For example, the absolute treated surface area of the subject can be the percentage of the collective treatment surface area of each target region together, relative to the entire body surface area of the subject. In some cases, the percentage of the absolute treated surface area to the total body surface area of the subject can be greater than or equal to 1, greater than or equal to 2, greater than or equal to 3.6, greater than or equal to 6.3, etc. In some cases, larger areas of the subject that are treated (e.g., greater than 1 percent) can elicit a greater metabolic response.

FIG. 2B shows a cross-section of the target region 130 of FIG. 2A, taken along line 2B-2B of FIG. 2A. As shown in FIG. 2B, the plurality of treatment regions 132 within the same row each extend through the epidermis 136 and the dermis 138 of the treatment region 130 of the skin tissue. In some cases, each of the treatment regions 132 can extend only through the epidermis 136 and only through the dermis 138. In other words, each of the treatment regions 132 do not extend into the superficial tissue 140 (e.g., that includes superficial fat). In this way, by avoiding thermal damage to the superficial tissue 140 can prevent undesirable lasting damage to the skin tissue at the target region 130 (e.g., poor wound healing, scarring, etc.). In some cases, each treatment region 132 can extend into a deep dermis of the dermis 132 of the target region 130, which can be thought to elicit a greater response (e.g., the deeper into the dermis 132 the thermal damage, the greater the elicited response). In some cases, the deep dermis can be the lower half of the dermis, the lower third of the dermis, etc. In some cases, each treatment region 132 can avoid extending through the epidermis at all. For example, when he energy source 110 is an ultrasound transducer, the energy 114 (e.g., the ultrasound energy) can be focused below the epidermis. In this way, the treatment regions 132 can be less visible when they heal (e.g., the treatment regions 132 may be less viable when viewed from an exterior surface, such as from a different person viewing the subject's skin tissue 112 that has been treated).

In some non-limiting examples, the one or more parameters can dictate the depth at which the treatment region 132 each extends into the skin tissue (e.g., the depth or length of the treatment region 132). For example, the total energy delivered to a treatment region 132 (e.g., the pulse energy of the laser) can determine the depth of the treatment region 132 (e.g., the maximum depth of the treatment region 132 into the skin tissue). Thus, in some cases, the computing device 106 can cause the energy source 110 to stop delivering the energy 114, when the desired amount of energy has been delivered, which can be indicate of the depth that a treatment region extends into the skin tissue. In other words, the computing device 106 can determine that the total energy delivered to a treatment region exceeds a maximum energy level (e.g., that is associated with a corresponding maximum depth), and the computing device 106 can stop the energy source 110 from delivering the energy 114, based on the total energy delivered having met or exceeded the maximum energy level.

FIG. 2C shows an example of a subject having multiple target regions that have been treated. For example, the chest of the subject has a first target region with a plurality of treatment regions (e.g., indicated as lines in FIG. 2C), and a second target region separated from the first target region that also includes a plurality of treatment regions.

FIG. 2D shows an example of a subject having a large target region that has been treated, while FIG. 2E shows an example of a subject having another large target region that has also been treated. For example, in FIG. 2D, the target region 145 that has a plurality of treatment regions 147 (e.g., indicated as lines in FIG. 2D) is located on the chest of the subject. For FIG. 2E, the target region 149 that has a plurality of treatment regions 151 (e.g., indicated as lines in FIG. 2E) is located on the back of the subject.

Referring back to FIG. 1, the energy source 110 can be implemented in many different ways to deliver the energy 114 to thermally damage the skin according to a pattern (e.g., an array of thermal damage, a fractional thermal damage pattern, a random pattern, etc.). For example, the energy source 110 can include one or more transducers that can convert energy from the power source 102 into a different form to be emitted out of the energy source 110 as the energy 114. As a more specific example, the transducer can be a light source (e.g., a laser) that can be configured to deliver a plurality of optical beams (e.g., laser beams), each of which create a respective treatment region in the target region of the skin tissue 112. In some cases, the optical beams can be delivered simultaneously, while in other cases, the optical beams can be delivered individually, separately, one at a time, multiple at a time, etc. For example, the optical beams can be delivered one row at a time, multiple rows at a time (e.g., the multiple rows being adjacent), one column at a time, multiple columns at a time (e.g., the multiple columns being adjacent), etc. In some cases, this can be implemented using a mask that is moveable that includes one or more holes (e.g., a slot, such as an elongated slot) that can allow one or more optical beams to pass through the mask at the one or more holes to the skin tissue 112 (e.g., to create a plurality of treatment regions), and the mask block one or more optical beams from passing through the mask to the skin tissue 112 (e.g., and thus not thermally damaging the skin with the one or more blocked optical beams). Then, the mask can be moved (e.g., by an actuator) to allow one or more different optical beams to pass through the mask at the one or more holes and the mask can block one or more different optical beams from passing through the mask to the skin tissue 112. In this way, each row(s), each column(s) of the treatment regions can be individually created one (or multiple) at a time. In some cases, by individually creating row(s), column(s), etc., of treatment regions, components of the treatment system 100 can advantageously made smaller (e.g., the energy source 110, such as when the energy source 110 is a laser), which can make the treatment system 100 easier to handle by a user, less heavy (e.g., when moving the treatment system 100), allows for smaller components (e.g., to decrease the cost of the treatment system 100), etc.

As another specific example, the energy source 110 can include one or more transducers that are ultrasound transducers, each of which can be configured to deliver therapeutic ultrasound energy. Thus, the energy 114 can be therapeutic ultrasound energy, which can create the plurality of treatment regions in the target region of the skin tissue 112. In some cases, the ultrasound transducers can be configured to emit high intensity focus ultrasound (“HIFU”). In some non-limiting examples, the energy source 110 can include an electrical generator (e.g., a waveform generator, an electrical signal generator, etc.), and the treatment system 100 can include one or more electrodes (e.g., an array of electrodes) electrically connected to the electrical generator, each of which can be a needle (e.g., a microneedle). In some cases, when an electrode is electrically excited and punctured into the skin tissue 112, the electrode can deliver a region of the energy 110 to the skin tissue 112 to create a treatment region. In some cases, the computing device 110 can selectively route electrical signals from the electrical generator (e.g., by opening or closing respective electrical switches) to select which electrode(s) receive electrical energy and which electrode(s) do not receive electrical energy (e.g. which the electrodes not receiving electrical energy corresponding to the non-treatment region). In some non-limiting examples, the puncture depth of an electrode, and the total energy delivered to the electrode can dictate the depth of the treatment region (e.g., in addition to the size of the electrode). Accordingly, in some cases, the treatment system 100 can deliver the energy 114 as radiofrequency (“RF”) energy.

In some non-limiting examples, rather than puncturing the skin tissue 112, the treatment system 100 can include a plurality of pins (e.g., each of which can be formed out of a metal), each of which can receive an electrical signal from the electrical generator of the energy source 110. Each pin that receives the electrical signal can be charged to a substantially high RF voltage. Then, when the plurality of pins are brought towards the target region of the skin tissue 112, a plasma can be created with the ambient environment (e.g., the atmosphere) to deliver plasma to the skin tissue 112 to form the plurality of treatment regions. In some cases, similarly to the electrode configuration above, the computing device 110 can selectively route electrical signals from the electrical generator (e.g., by opening or closing respective electrical switches) to select which pin(s) receive electrical energy (e.g., corresponding to the formation of a treatment region) and which electrode(s) do not receive electrical energy (e.g. which the pins not receiving electrical energy corresponding to non-treatment region). In some non-limiting examples, the voltage provided to a pin, the duration of charging of the pin, the distance the pin is from the skin tissue 112, the duration of discharge of the voltage from the pin (e.g., corresponding to a total energy delivered from a pin to the skin tissue 112 to form a treatment region), etc., can dictate the size of the treatment region (e.g., the depth, the width, etc.). Accordingly, in some cases, the treatment system 100 can deliver the energy 114 according to fractional micro plasma radiofrequency.

In some non-limiting examples, the treatment system 100 can deliver thermal energy (e.g., similar to a thermal mechanical skin rejuvenation system), which can be implemented without the use of laser, and without ablating the skin tissue 112. For example, the treatment system 100 can include a plurality of thermally conductive tips (e.g., in an array), which can be selectively heated in a similar manner as the other configurations (e.g., with each tip being in thermal communication with one or more heaters, which an be a resistive heater). When a tip is heated, and is brought into contact with the skin tissue 114, a treatment region is created. Correspondingly, tips that are not heated and are brought into contact with the skin tissue 114 correspond to non-treatment region of skin tissue. In some non-limiting examples, the amount of contact between a tip and the skin tissue 112 can determine the depth, the cross-sectional area, etc., of a treatment region. In addition, the temperature of the tip (e.g., which can be caused by the energy source 110 heating, such as electrically heating, the tip, which can depend on the electrical signal provided to the electrical heater), can also determine the depth, the cross-sectional area, etc., of a treatment region.

In some non-limiting examples, and advantageously the treatment regions described herein can be created in various ways without puncturing the skin tissue, which can otherwise undesirably compromise the integrity of the epidermis and thus increase the likelihood of infections.

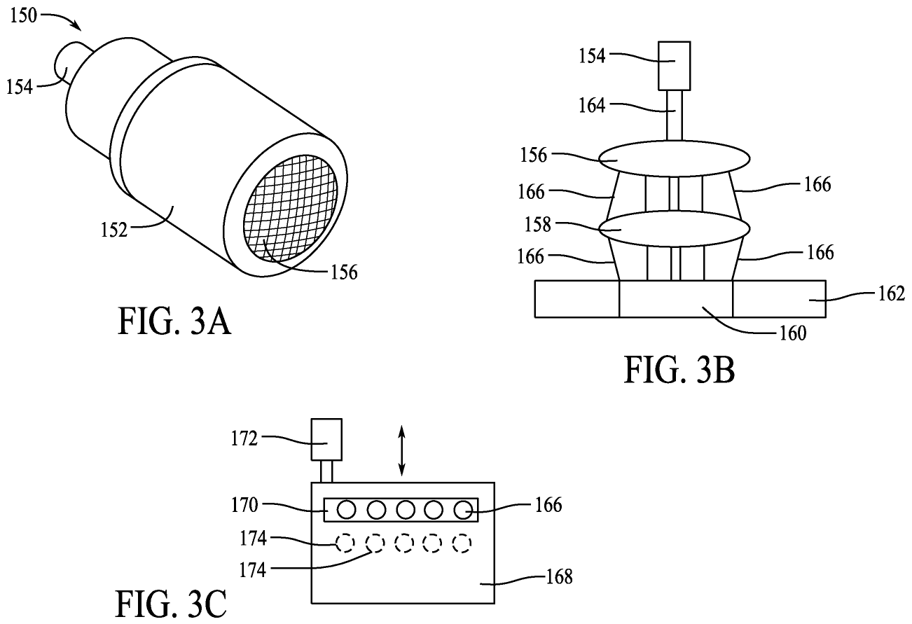

FIG. 3A shows a schematic illustration of a treatment system 150, which can be a specific implementation of the treatment system 100. Thus, the treatment system 150 pertains to the treatment system 100 (and vice versa). The treatment system 150 can be configured to emit energy to create a plurality of treatment regions of a target region of skin tissue and a non-treatment region of skin tissue. The treatment system 150 can include a housing 152, a laser 154, and a lens 156 that is configured to split the laser beam from the laser 154 into a plurality of separate laser beams. For example, the lens 156 can be a pixel beam splitting lens, which can split the laser beam emitted from the laser 154 into a plurality of individual laser beams. In this way, each individual laser beam (e.g., split from the initial laser beam) can create a respective treatment region in the target region of the skin tissue.

FIG. 3B shows a cross-sectional view of the treatment system 150 with respect to a target region 160 of skin tissue 162. In some configurations, the treatment system 150 can include a focusing lens 158 optically coupled to the lens 156, which can focus the individual laser beams after being split. As shown in FIG. 3B, the laser beam 154 can emit a laser beam 164 towards the lens 156, which can split the laser beam 164 into a plurality of laser beams 166. The laser beams 164 can be focused by the focusing lens 158 and can be directed at the skin tissue 162 to each create a respective treatment region within the target region 160 of the skin tissue 162. In some cases, as described above, peripheral treatment regions can define the boundary of the target region 160. Correspondingly, peripheral laser beams (e.g., the laser beams 166) can define the periphery of a field of treatment that aligns with the target region 160.

In some non-limiting examples, while the treatment system 150 has been described as having a single laser 154, in other configurations, the treatment system 150 can have multiple lasers that can be selectively activated (e.g., by a computing device) to selectively emit different laser beams to selectively create different treatment regions at different times. In this way, the treatment system 150 can scan (e.g., similar to a raster scan) the treatment regions onto the skin tissue 162 in a sequential manner (e.g., each row, each column, etc.). This scanning configuration can be implemented in different ways. For example, FIG. 3C shows an illustration of a shield 168 that can include a slot 170, and an actuator 172 (e.g., a linear actuator) coupled to the shield 168. As shown in FIG. 3C, the slot 170 can allow passage of some of the laser beams 166, while the shield 168 can block other laser beams 174 (e.g., also split from the laser beam 164) from passing through the shield 166. Then, after the treatment regions have been created using the laser beams 166, the shield 168 can be moved by the actuator 172 (e.g., translated in a direction substantially perpendicular to the elongation of the slot 170) to allow the laser beams 172 to pass through the hole 168, while blocking the laser beams 166. In this way, the treatment system 150 (e.g., a computing device) can cause the actuator to sweep laser beams across the treatment region 160 of the skin tissue 162 to effectively scan the treatment regions on the skin tissue 162. As another example, this sweeping can be implemented using different optical components that can focus different laser beams onto different regions of the skin tissue 162. For example, an actuator can move different lenses (or other optical components, such as prisms, mirrors, etc.) into and out of the path of the laser beams 166 to direct the laser beams 166 at different regions of the skin tissue to scan the treatment regions onto the target region 160 of the skin tissue 162. As yet another example, in an alternative configuration, the treatment system 150 can include a first optical fiber, an optical fiber splitter optically coupled to the optical fiber, and a plurality of optical fibers optically coupled to the optical fiber. In this way, the laser 154 can direct the laser beam 164 along the first optical fiber, until the laser beam 164 is split by the optical fiber splitter and the plurality of laser beams propagate along a respective optical fiber of the plurality of fibers to be delivered to the target region 160 of the skin tissue 162. In this way, the treatment system 150 can be translated (e.g., by an actuator) until the treatment regions have been created.

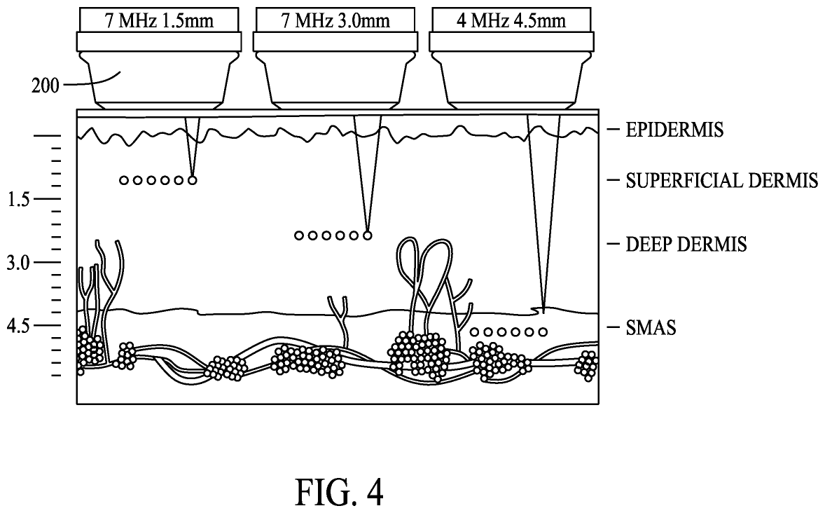

FIG. 4 shows a schematic illustration of a treatment system 200, which can be a specific implementation of the treatment system 100. Thus, the treatment system 200 pertains to the treatment system 100 (and vice versa). The treatment system 200 can be a therapeutic ultrasound system, which can include one or more ultrasound transducers (e.g., a piezoelectric transducer), each of which can be configured to deliver therapeutic ultrasound energy to create one or more treatment regions within the treatment target. For example, the one or more ultrasound transducers can be a plurality of ultrasound transducers (e.g., within an array) with at least one ultrasound transducer (or multiple ultrasound transducers) delivering therapeutic ultrasound energy to create a therapeutic region (e.g., a single therapeutic region) within the target region of the skin tissue. As shown in FIG. 4, the frequency of the therapeutic ultrasound energy (e.g., the frequency of the electrical signal used to drive an ultrasound transducer) can dictate the depth that a treatment region extends into the skin tissue. For example, an ultrasound transducer that emits therapeutic ultrasound having a frequency of 4 megahertz (“MHz”), can cause a treatment region to extend into the superficial tissue (e.g., extend to a depth of 4.5 millimeters). Thus, a computing device of the treatment system 200 can cause each ultrasound transducer to operate at a frequency greater than 4 MHZ (e.g., because increasing frequency is inversely related to depth of the treatment region, with deeper treatment regions being cause by lower frequencies and vice versa).

In some non-limiting examples, the therapeutic ultrasound system can deliver high frequency ultrasound in with frequency in the MHz range and with focusing of the dermal tissue in a range between 0 mm and 4 mm. The size of a focal zone generated by a HIFU transducer is inversely dependent on the operating frequency; that is, the higher the frequency, the smaller the focal zone. In some cases, for HIFU treatments to be reliable in dermatology, the focal zone should be positioned and confined accurately within the epidermis, dermis, or subcutaneous depending of the purpose of the target and the desired intervention. As demonstrated in the testing, accuracy in skin targets may require an operating frequency of approximately 4-20 MHz.

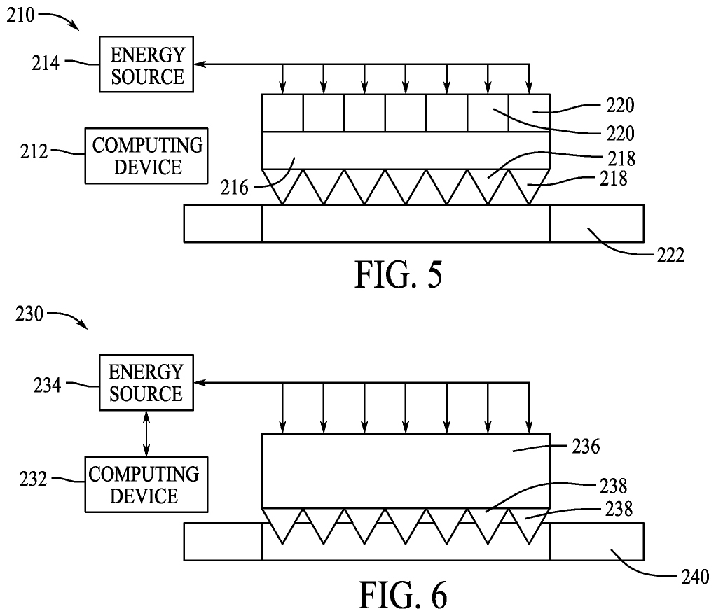

FIG. 5 shows a schematic illustration of a treatment system 210, which can be a specific implementation of the treatment system 100. Thus, the treatment system 210 pertains to the treatment system 100 (and vice versa). The treatment system 210 can be a thermo-mechanical fractional injury (“TFMI”) device, which is a non-laser, fractional, non-ablative, thermomechanical skin rejuvenation system, which combines thermal energy with motion. The treatment system 210 can include a computing device 212, an energy source 214, a substrate 216 including a plurality of tips 218 (e.g., thermally conductive tips), and a plurality of electrical heaters 220 (or one electrical heater). The plurality of tops 218 can include two types of tips which can be a standard tip including 81 (9×9) or other numbers of tiny titanium pyramids, and a small tip (also known as a periorbital tip) including of 24 (6×4) tiny pyramids. Each tip 218 can be pyramidal in shape, and can be relatively small (e.g., less than 1000 μm in length). The tip base can be heated to a temperature (e.g., 400° C.) within a handpiece, which quickly moves toward the skin surface to achieve contact and coagulate tissue, creating microcraters by evaporation and desiccation. The amount of thermal energy delivered to the skin can determined by the pulse duration (PD; range: 5-18 milliseconds) in which the tips 218 are actually in contact with the skin tissue 222 to deliver the thermal energy, and the protrusion distance or depth (100-1000 μm) can be the amount of surface area contact between a tip 218 and the skin tissue 222 (e.g., the protrusion distance is the distance the heated tip projects from the edge of the handpiece gauge per actuation). In other words, the axial distance between the substrate 216 and the skin tissue 222 can also be the protrusion distance (e.g., with smaller axially distances causing greater thermal damage and thermal damage depth and vice versa). Accordingly, a greater protrusion distance leads to a greater degree of skin contact between the titanium pyramids, fewer air gaps, and greater thermal transfer. Importantly, thermal transfer in TMFI technology does not involve any mechanical penetration of the epidermis. In some cases, the treatment system 210 can include an actuator coupled to the substrate 216 to selectively bring the tips 218 into (and out of) contact with the skin tissue 222.

In some non-limiting examples, the computing device 212 can cause the energy source 214 (e.g., an electrical generator) to selectively turn on particular heaters 220 (e.g., a resistive heater), each of which is in thermal communication with a respective tip 218. In this way, a pattern of thermal damage can be implemented accordingly. In addition, each tip that is heated can create a respective treatment region in the skin tissue 222.

FIG. 6 shows a schematic illustration of a treatment system 230, which can be a specific implementation of the treatment system 100. Thus, the treatment system 230 pertains to the treatment system 100 (and vice versa). The treatment system 230 can be an RF device, and can include a computing device 232, an energy source 234, a substrate 236, an a plurality of needles 238 (e.g., a 2D array of needles including multiple rows of needles, multiple columns of needles, etc.). The plurality of needles 238 can each include a pair of electrodes. As shown in FIG. 6, the substrate 236 can be brought towards the skin tissue 240 (e.g., using an actuator), until the needles 238 penetrate the skin tissue 240. Then, the energy source 234 (e.g., an electrical generator) can generate an electrical signal to charge each of the electrodes of the needles 238. The electrical signal can be in a range between 3 kHz to 300 MHz, while in other cases the electrical signal can be in a range between 0.5 MHz to 40 MHz. In some cases, the frequency of the electrical signal for the RF device can be inversely proportional to the depth of penetration of creation of a treatment region. For example, lower frequencies can have higher penetration rates and vice versa. Similarly to the treatment system 210, each needle 238 that is electrically excited can create a respective treatment region in the skin tissue 222. In some configurations, the parameters that can relate to the creation of the treatment regions in the skin tissue 240 can be the needle penetration depth, the conduction times, the energy level delivered to an electrode (e.g., voltage applied across the electrode, the pulse width of the voltage, etc.), each of which can significantly affect dermal coagulation.

In some non-limiting examples, the treatment system 230 can also be a fractional micro plasma RF device. In this case, the needles 238 do not have to penetrate the skin tissue 240 to deliver the energy to create the treatment regions. Rather, each needle 238 can be replaced with a respective pin and each pin can be charged using the electrical signal. The substrate 236 can be moved towards the skin tissue 240, and without the pins contacting the skin tissue 240, the pins that have been charged can be discharged (e.g., via plasma discharge) to create the treatment regions (e.g., with each charged pin creating a respective treatment potion in the skin tissue 240).

Referring back to FIG. 1, the treatment system 100 can include one or more shields 120 that can be configured to cover a sensitive area to prevent thermal damage of tissue at the sensitive area. For example, a shield can be eyewear (e.g., glasses, goggles, etc.), garments (e.g., clothes), which can cover sensitive areas (e.g., eyes, the groin, etc.). In some cases, the shield 120 can be placed over the sensitive area and can absorb, reflect, etc., the energy 114 to avoid thermally damaging the skin tissue underneath the shield 120.

In some non-limiting examples, the treatment system 100 can include one or more sensors 116, one or more imaging systems 118, etc., that can be used to determine the one or more parameters of the energy source 110, to avoid treating particular targets (e.g., sensitive areas), or to ensure that a desired target region has been treated. The sensors 116, and the imaging systems 118 can be in communication with the computing device 106. In some non-limiting examples, the sensor 116 can include a distance sensor (e.g., a time of flight sensor), an image sensor (e.g., a camera), etc. For example, the distance sensor can receive a current distance between the energy source 110 and the skin tissue 112, which can be used to adjust the one or more parameters of the energy source 110. For example, when the energy source 110 is a laser the farther the energy source 110 is away from the skin tissue 112 the less power can be delivered to the skin tissue 112 to create the treatment regions. So, the distance can be used to increase (or decrease) the power of the energy source 110. As another example, an image sensor can acquire an image of the skin tissue 112 (e.g., at the target region), which can be prior to the delivery of the energy 114. In this way, the computing device 106 can determine a skin tone (or melanin content) of the skin tissue 112 (e.g., at the target region) to compensate for the skin tone of the subject. For example, melanin at the epidermis can act as a chromophore, which absorbs more laser energy, which can increase the risk of epidermal injury for individuals with darker complexions. Thus, the one or more parameters can be adjusted to compensate for skin tone, which can include a long pulse of laser energy (over a short pulse of laser energy) in which the long pulse has a smaller amplitude than the short pulse to deliver a more controlled energy to the skin tissue. In addition, the one or more parameters can include a low fluence (over a high fluence) for darker skin tones, and a low density of the thermal regions (rather than a high density) for darker skin tones.

In some non-limiting examples, the imaging system 118 can be an ultrasound imaging system (e.g., an ultrasound imaging device), an optical coherence tomography imaging system, a photoacoustic imaging system, etc., which can acquire imaging data from the skin tissue 112 and determine a skin thickness (e.g., thickness of the epidermis) using the imaging data. Then, the computing device 106 can determine (or change) the one or more parameters of the energy source 110, based on the skin thickness. For example, determining the skin thickness can ensure that the subcutaneous tissue is not reached by the treatment regions, and the energy to be delivered to each treatment region can be determined based on the thickness of the skin. For example, the laser power (e.g., energy to be delivered to a treatment region) should be increased, the distance between the energy source 110 and the skin tissue 112 should be decreased, etc., for thicker epidermises (and vice versa). In this way, the treatment regions are ensured to extend to the desired depth into the dermis.

In some non-limiting examples, the imaging system 118 can be an image sensor (e.g., as part of a camera, which can be, for example, a CCD, a 3D camera, etc.). The image sensor can acquire one or more images of the subject and the computing device 106 can generate a 3D volume of the subject. Then, the computing device 106 can locate sensitive regions to avoid on the 3D volume (e.g., the groin), and can locate areas to target on the 3D volume. For example, the computing device 106 can receive a user input from the user input device 108 to mark one or more areas on the 3D volume to target to target, and one or more area on the 3D volume to avoid. Then, the computing device 106, can register the 3D volume of the subject to the energy source 110 (e.g., with the coordinate system of the energy source 110 registered with the coordinate system of the imaging system 118) to ensure that the targeted regions of the 3D volume are treated with the energy 114, and that the one or more areas to avoid are not treated with the energy 114. In some cases, while a 3D volume of the subject has been described, the 3D volume can be replaced with a 2D view of the subject. In some non-limiting examples, one or more user inputs from the user input device 108 can be indicative of the data from the sensors 116 or the imaging systems 118. For example, the computing device 106 can receive a user input indicative of at least one of a skin tone of the subject, a skin thickness of the subject (e.g., including the thickness of an dermis of the subject), etc.

In some non-limiting examples, including after a target region has been identified, the one or more parameters of the energy source 110 have been determined, the shields 120 have been placed on the subject, etc., the computing device 106 can cause the energy source 110 to deliver the energy 114 to the skin tissue 112 to create the plurality of treatment regions in the target region. In some cases, the computing device 106 can sequentially create the plurality of the treatment regions (e.g., using a region of the energy 114 as a burst). For example, a first region of the energy 114 can be delivered to create a first subset of the plurality of treatment regions, then a second region of the energy 114 can be delivered to create a second subset of the plurality of treatment regions, and so on, until the entire target region has been scanned.

In some non-limiting examples, the energy 114 can treat a single target region of the skin tissue 112 of the subject. However, in other cases, the treatment system 100 can treat multiple different target regions. For example, after the energy 114 has been delivered to the skin tissue 112 to create the plurality of treatment regions in a first target region of the skin tissue 112, the treatment system 100 can be moved to a different location (e.g., the energy source 110 can be moved, such as by a robot arm) to deliver other energy from the energy source 110 to create a plurality of treatment regions in a second target region of the skin tissue 112 (e.g., different from the first target region).

In some non-limiting examples, the one or more parameters of the energy source 110 can create a plurality of treatment regions in the skin tissue 112 at one or more target regions of the skin tissue 112, which can elicit a response in the subject. In some cases, the response can be increasing the metabolism (e.g., substantially increasing the metabolism) of the skin tissue at the one or more target regions or a different region of the skin tissue 112 that does not include a treatment region (e.g., on a different extremity as the one or more target regions, on a different side as the one or more target regions, etc.). In some cases, the response can be (substantially) increasing a basal metabolic rate of the subject. In some non-limiting examples, the response can be decreasing an amount of fat (e.g., white adipose tissue) at the one or more target regions or a different region of the skin tissue 112 that does not include a treatment region (e.g., on a different extremity as the one or more target regions, on a different side as the one or more target regions, etc.). In some cases, the response can be (substantially) decreasing a thickness of fat at the one or more target regions or a different region of the skin tissue 112 that does not include a treatment region (e.g., on a different extremity as the one or more target regions, on a different side as the one or more target regions, etc.). In some cases, the response can be (substantially) decreasing a total amount of fat of the subject. In some cases, the response can be (substantially) decreasing a total weight of the subject, which can be without (substantially) decreasing a total lean mass of the subject. In some non-limiting examples, the response can be transforming one or more white fat cells (e.g., at a target region of the skin tissue 112 or a region of the skin tissue that does not include treatment regions) into a beige fat cell or a brown fat cell. In some cases, the response can be (substantially) increasing the concentration of a hormone in the subject, which can be noradrenaline. In some cases, the response can be (substantially) increasing the concentration of an immune modulator, an immune system protein, a pro-inflammatory protein, a cytokine, which can be IL-6.

In some non-limiting examples, the response can be treating, alleviating, improving, etc., one or more diseases associated with a weight disorder (e.g., obesity, being overweight, etc.). For example, the weight disorder can be responsible for causing the one or more diseases. Thus, with an (substantial) improvement in the weight disorder, the one or more diseases can be improved. For example, the one or more diseases can be diabetes (e.g., type two diabetes), insulin resistance, high blood pressure, heart disease, mental illness, pain (e.g., in one or more joints from being overweight or obese), high levels of cholesterol, high triglyceride levels, etc. Thus, when the weight disorder is improved (e.g., by decreasing the total amount of fat), the one or more diseases caused by the weight disorder can be improved.

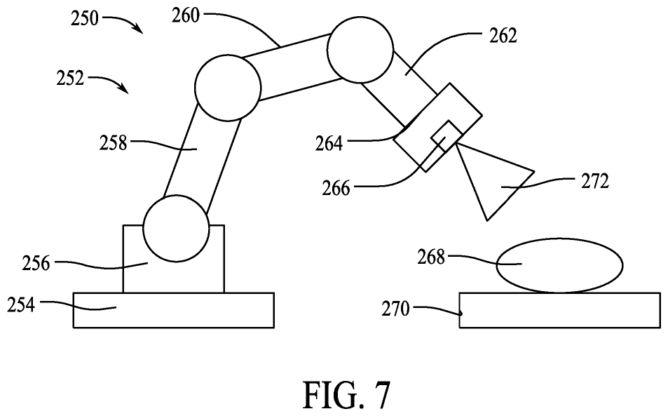

FIG. 7 shows a schematic illustration of a treatment system 250, which can be a specific implementation of the treatment system 100. Thus, the treatment system 250 pertains to the treatment system 100 (and vice versa). The treatment system 250 can cover large swaths of a subject, which can be important for eliciting the desired metabolic response, or response associated with the metabolic function of the subject. For example, the treatment system 250 can include a robot arm 252, which can be a multi-axis robot having one or more degrees of freedom (e.g., one, two, three, four, five, six, seven degrees of freedom, etc.). In some cases, the greater the number of degrees of freedom up until a certain point (e.g., six degrees of freedom) can increase the maneuverability of the robot arm 252 and allow the robot arm 252 to reach more skin areas of the subject. The robot arm 252 can include a support structure 254 (e.g., which can support the robot arm 252 relative to the subject), a base 256 that is rotatable, an arm 258 pivotally coupled to the base 256, an arm 260 pivotally coupled to the arm 258, an arm 262 pivotally coupled to the arm 260, and an end effector 264 coupled to the arm 262 (e.g., at the opposing end of the arm 262).

As shown in FIG. 7, the treatment system 250 can include an energy source 266 that can be coupled to the end effector 264 (e.g., or otherwise integrated within the end effector 264). However, in alternative configurations, the energy source 266 can be coupled to a different location of the robot arm 252. A subject 268 can be supported on a table 270, which can be adjacent to the robot arm 250. In some non-limiting examples, a computing device of the treatment system 250 can control the robot arm 252 and the energy source 266 to deliver energy to the skin tissue of the subject 268. For example, a computing device can determine, receive, etc., a scanning routine for treating one or more target regions of the skin tissue of the subject 268 (e.g., according to the scanning routine). In this way, the computing device can implement the scanning routine to create a plurality of treatment regions in the skin tissue of the subject 268 according to a scanning routine. For example, this can include a computing device moving the robot arm 252 and the energy source 266 (e.g., that is coupled to the robot arm 252) to a first target region, stopping the robot arm 252 at the first target region, delivering the energy 272 from the energy source 266 to the first target region (e.g., while the robot arm is stopped), moving the robot arm 252 and the energy source 266 to a second target region (e.g., that is different than the first target region), stopping the robot arm 252 at the second target region, delivering the energy 272 from the energy source 266 to the second target region (e.g., while the robot arm is stopped), and so on, until all of the desired target regions of the subject 268 have been treated.

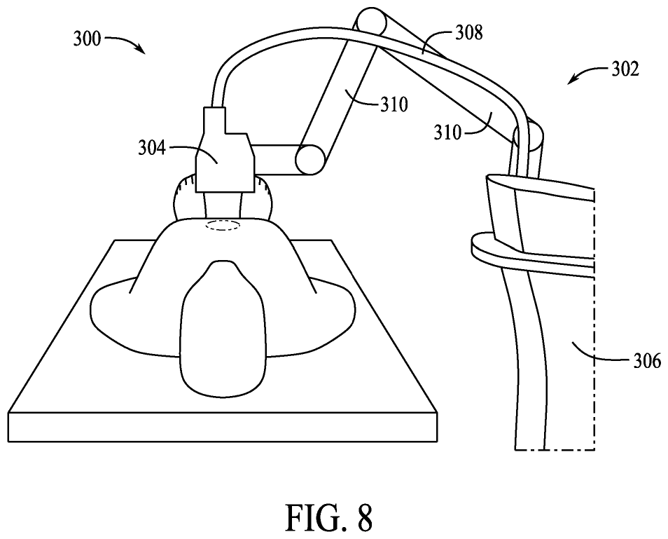

FIG. 8 shows a schematic illustration of a treatment system 300, which can be a specific implementation of the treatment system 100. Thus, the treatment system 300 pertains to the treatment system 100 (and vice versa). The treatment system 300 can also cover large swaths of a subject, which can be important for eliciting the desired metabolic response, or response associated with the metabolic function of the subject. The treatment system 300 can include a support structure 304, and an energy source 302 coupled to the support structure 304 (e.g., at an end of the support structure 304). The support structure 304 can include a base 306 (e.g., that can include a power source to power the energy source 302, such as via a cable 308, or an optical fiber), and a plurality of linkages that can be lockable. For example, each linkage 310 can include a lock (e.g., that is rotatable to lock pivoting of the linkage, and rotatable in the opposing direction to allow pivoting of the linkage). In this way, a user can move the energy source 304 with the linkages unlocked to a desired position, and then can subsequently lock the linkages 310 to lock the desired position of the energy source 304.

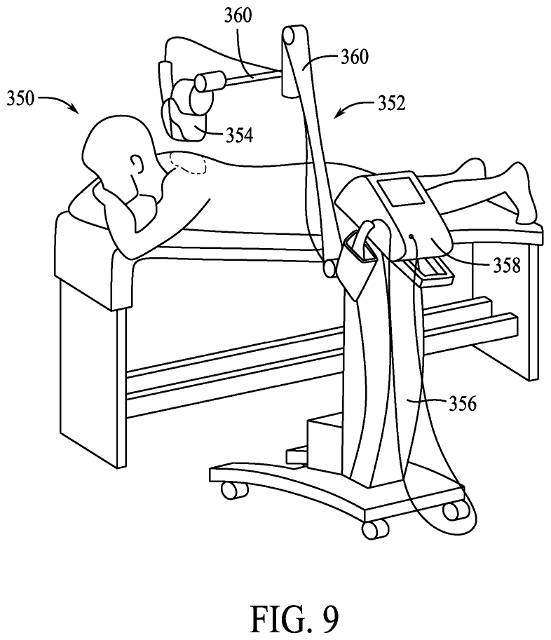

FIG. 9 shows a schematic illustration of a treatment system 350, which can be a specific implementation of the treatment system 100. Thus, the treatment system 350 pertains to the treatment system 100 (and vice versa). The treatment system 350 can also cover large swaths of a subject, which can be important for eliciting the desired metabolic response, or response associated with the metabolic function of the subject. Similarly to the treatment system 300, the treatment system 350 can include a support structure 352 and an energy source 354 coupled to the support structure 352. The support structure 352 can include a base 356, a user input device 358 (e.g., a touchscreen) that can be coupled to the base 356, and a plurality of linkages 360 that can be lockable to support the energy source 354 relative to the patient. In some cases, the support structure 352 can include one or more wheels, slides, etc., to move the support structure 352 relative to the subject.

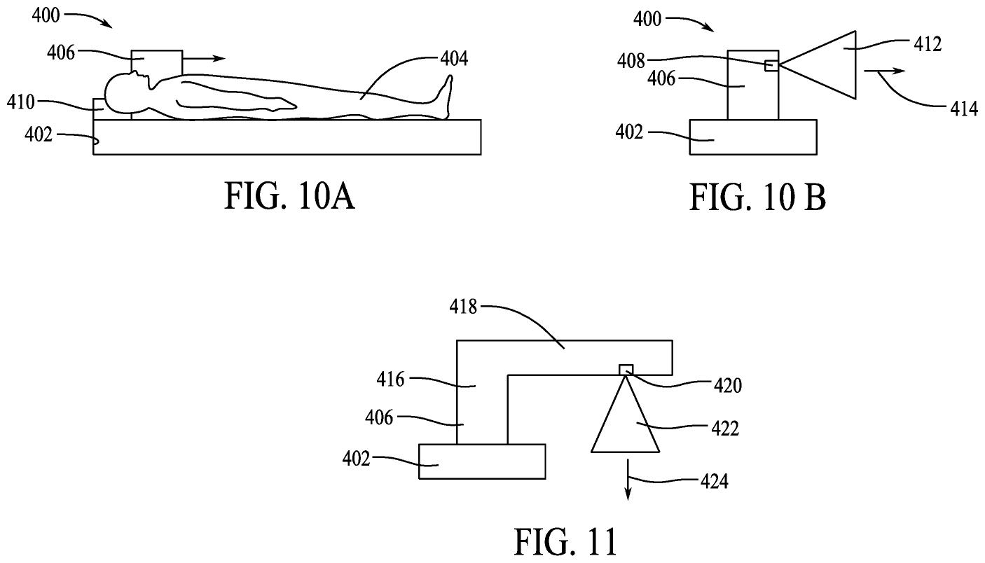

FIG. 10A shows a side view of a schematic illustration of a treatment system 400, while FIG. 10B shows a front view schematic illustration of the treatment system 400. The treatment system 400 can be a specific implementation of the treatment system 100. Thus, the treatment system 400 pertains to the treatment system 100 (and vice versa). The treatment system 400 can also cover large swaths of a subject, which can be important for eliciting the desired metabolic response, or response associated with the metabolic function of the subject. The treatment system 400 can include a table 402 that can support a subject 404, a slide 406, an energy source 408 coupled to the slide 406, and an actuator 410 coupled to the slide 406 (and the table 402). The actuator 410, which can be a rotational actuator (e.g., a motor), a linear actuator, etc., can be configured to move the slide 406 (and thus the energy source 408) along the table 402 in a first direction and a second direction opposite the first direction. In this way, the energy source 408 can be brought into alignment with different regions of the subject 404, so that energy 412 from the energy source 408 can be directed to different target regions of the subject. As shown in FIG. 10B, the energy 412 can be emitted towards the subject along a direction 414 that is substantially perpendicular to the first direction and second direction of movement of the slide 406. In some cases, the configuration of the slide 406 in FIGS. 10A and 10B can target side surfaces of the subject (e.g., when the subject is laying on their back), or other surfaces with different positioning of the subject.

FIG. 11A shows a front schematic view of an alternative configuration of the slide 406. In this configuration, the slide 406 includes a first region 416 that longitudinally extends along a first direction (e.g., substantially perpendicular to the movement direction of the slide 406), and a second region 418 coupled to the first region 418 and that longitudinally extends along a second direction (e.g., substantially perpendicular to the movement direction of the slide and substantially perpendicular to the direction in which the first region 418 longitudinally extends). As shown in FIG. 11A, thee energy source 420 can be coupled to the second region 418. In this way, the second region 418 and the energy source 420 can be positioned above the subject, so that the energy 422 delivered by the energy source 420 is directed downwardly towards the subject along a direction 424 (e.g., that is substantially perpendicular to the direction of movement of the slide 406).

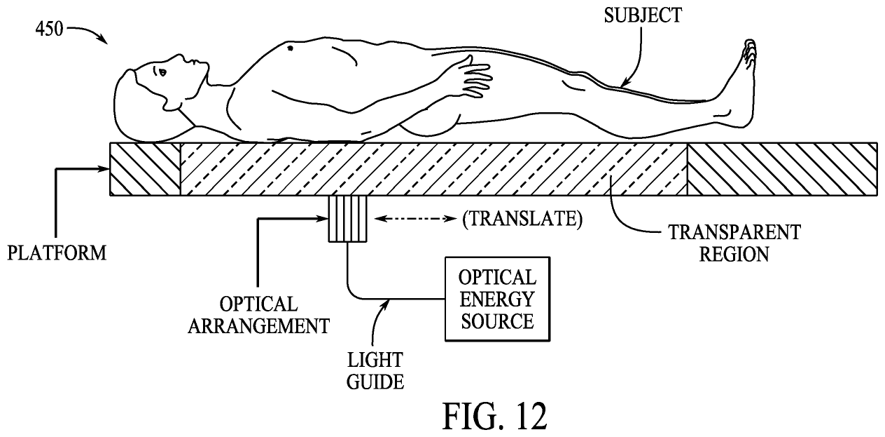

FIG. 12 shows a side view of a schematic illustration of a treatment system 450, which can be a specific implementation of the treatment system 100. Thus, the treatment system 450 pertains to the treatment system 100 (and vice versa). The treatment system 450 can also cover large swaths of a subject, which can be important for eliciting the desired metabolic response, or response associated with the metabolic function of the subject. The treatment system 450 can include a platform, a laser or other optical energy source, and an optical arrangement configured to direct optical energy onto a subject lying on the platform.

The platform can be at least partially made of an optically transparent substance, such as a glass or certain plastics. In use, the subject can lie down on the platform. The optical arrangement can be configured to direct multiple beams of optical energy through the optically transparent region of the platform and onto a region of the subject's back. Such energy can be directed to generate a fractional pattern of thermal damage or ablation in the skin of the subject.

In some non-limiting examples, a light-transmitting substance such as, e.g., glycerin or the like, can be provided between the platform and the subject's skin. The substance can be provided on the platform or applied topically to the treatment area prior to treatment. The presence of such substance can reduce mismatches and transitions in refractive indices, and thus improve the optical pathway between the optical arrangement and the skin. For example, reflection and/or scattering of the optical energy being delivered may be reduced, such that the optical energy remains more focused and less energy is lost to such phenomena as surface scattering.

In some non-limiting examples, the substance can include a pain-killing, numbing, or analgesic compound. Such compound can reduce the amount of pain or discomfort that may be felt during the application of optical energy to the skin.

In further non-limiting examples, cooling or pre-cooling of the skin regions being irradiated can be provided. Such cooling can be provided, e.g., by spray cooling and/or contacting the skin surface with a cooled object (e.g. a pre-cooled or actively cooled plate or block of material) prior to laser exposure.

In one non-limiting example, the optical arrangement can include one or more rows of spaced-apart optical fibers, where the ends of such fibers are directed toward the skin of the subject lying on the platform. Such fibers can be used to direct optical energy from the laser or optical energy source onto the skin, acting as light guides. Small lenses may optionally be used to reduce the beam diameters to a width of about 1 mm or less.

In some non-limiting examples, the optical arrangement can be provided with a translating arrangement, such that the optical arrangement can be scanned in one or two directions along the platform, parallel to the lower surface thereof. For example, energy to one or more rows of optical fibers in the arrangement can be pulsed, and the optical arrangement translated during such pulsed energy delivery (e.g., between pulses), to generate a fractional pattern of optical energy applied to the skin of the subject.

In some non-limiting examples, the optical arrangement can be translated over a particular region of the subject a plurality of times, in the x- and/or y-directions, to produce a fractional pattern of delivered energy having the desired density or fractional surface coverage.

In other non-limiting examples, a single laser spot can be pulsed and scanned over a region of the subject's skin to produce a fractional damage pattern, although such single-spot translation may lead to longer treatment times as compared to simultaneous application of energy using a plurality of light fibers or other light guides.

In yet a further non-limiting example, the platform may include one or more cutout areas (e.g., one or more holes), and the optical arrangement can be configured to directly contact the skin surface of the subject within the cutout area. Again, the optical arrangement can include a single pulsed energy beam, or a one- or two-dimensional array of beams, which may be produced by a plurality of light guides.

With this exemplary non-limiting example, a subject can also be positioned on their side or stomach, and further regions of the skin surface can be treated with optical energy to generate a fractional pattern of thermal damage and/or ablation over a large region of skin.

In yet another non-limiting example, optical arrangements as described herein can be provided both above and below a subject simultaneously, such that front and back body regions can be irradiated with fractional patterns of energy simultaneously. Further, if two or more separate regions of the body are being treated simultaneously, the individual damage regions may be spaced further apart than what is commonly done in cosmetic fractional treatments. For example, successively or simultaneously generated thermal injury spots can be spaced about 0.5 cm or a centimeter apart during treatment. Such spaced-apart damage can be well-tolerated and generate a lesser degree of pain as compared to fractional treatments where simultaneous or near simultaneous damage zones are generated much closer together. For example, during cosmetic fractional treatments, simultaneous damage zones are created that are typically less than about 1 mm apart. Higher local area coverages can be achieved by performing multiple passes of the fractional damage process over a single region. For non-limiting examples using thermal damage techniques, the wider spacing of individual spots can also facilitate more localized cooling and thermal recovery of the skin tissue prior to subsequent passes being made over the same region. In this manner, pain and discomfort can be reduced while also avoiding unwanted thermal buildup and damage in the treated regions.

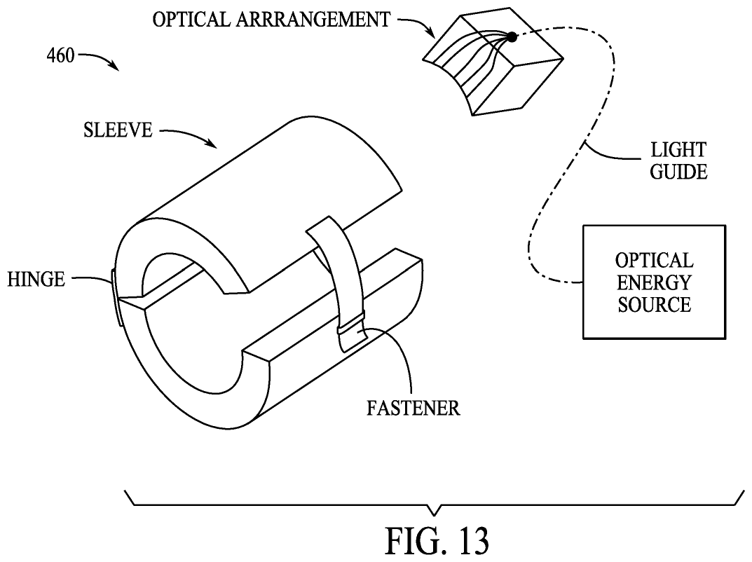

FIG. 13 shows a side view of a schematic illustration of a treatment system 460, which can be a specific implementation of the treatment system 100. Thus, the treatment system 460 pertains to the treatment system 100 (and vice versa). The treatment system 460 can also cover large swaths of a subject, which can be important for eliciting the desired metabolic response, or response associated with the metabolic function of the subject. The treatment system 460 can include a rigid or semi-rigid sleeve arrangement, a laser or other source of optical energy, and an optical arrangement. The sleeve can include two or more rounded sections (e.g., pivotally coupled to each other), configured to wrap at least partially around a region of the subject, including a limb (e.g., an arm or a leg), a torso of a subject, etc. The sleeve can be provided with a hinge and a fastener (e.g., a flexible or adjustable fastener), such that the sleeve can be attached over at least a region of an arm or a leg (e.g., with a leg positioned within the sleeve, the sleeve can be locked using the fastener). The sleeve can be made at least partially of an optically transparent substance, such as a glass or certain plastics (or a hole can be directed through a region of the sleeve to receive the optical arrangement or optical energy source). In some non-limiting examples, at least a region of the sleeve can be bendable or flexible to provide better conformance to the size and/or shape of the limb.

In use, the sleeve can be secured over at least a region of the subject's limb. The optical arrangement can be configured to direct multiple beams of optical energy through the optically transparent region of the sleeve and onto a region of the subject's limb. Such energy can be directed to generate a fractional pattern of thermal damage or ablation in the skin of the subject.

A light-transmitting substance such as, e.g., glycerin or the like, can be provided between the sleeve and the subject's skin to improve the optical pathway between the optical arrangement and the skin, as described herein. In some non-limiting examples, the substance can include a pain-killing, numbing, or analgesic compound to reduce the level of potential discomfort that may be felt during the procedure.

In some non-limiting examples, the optical arrangement can include a one- or two-dimensional array of optical fibers or other light-emitting elements. The arrangement can be configured, e.g., with a concave cylindrical profile that conforms to the outer surface of the sleeve. The optical arrangement can be more generally configured to translate over at least a region of the sleeve, longitudinally and/or rotationally. Pulsed light from the optical energy source can be combined with the translation speed and pattern, and spacing of the light-emitting elements, to generate a fractional pattern of thermal damage or ablation on the skin of the limb being treated. Optionally, the optical arrangement can be translated a plurality of times over a single region of the skin to generate denser patterns of damage having a larger surface fraction of irradiated tissue.

In some non-limiting examples, longitudinal and/or circumferential guides or tracks can be provided on the sleeve to direct the optical arrangement along certain paths. Such guides or tracks can facilitate uniform translation of the optical arrangement in longitudinal and/or circumferential directions to provide more control over the fractional irradiation patterns produced.

In further non-limiting examples, a single laser spot can be pulsed and scanned over a region of the subject's limb to produce a fractional damage pattern, although such single-spot translation may again lead to longer treatment times as compared to using a plurality of light fibers or other light guides.

In still further non-limiting examples, the optical arrangement can be provided as a contoured handheld device configured to be translated by hand over at least a region of the sleeve. Directing pulsed energy through the optical arrangement onto the skin during such translation can thus generate a fractional pattern of irradiation.

In some non-limiting examples, the sleeve can be omitted and the optical arrangement can be provided with a contoured or flexible surface to facilitate manual translation of the optical arrangement over different regions of a subject's skin.

In any of the non-limiting examples described herein, an orientation, position, speed, and/or velocity sensor can be provided on the optical arrangement, and configured to detect an orientation, position, and/or speed of the optical arrangement relative to the subject's skin. Such sensor can be coupled to a control arrangement for the optical energy source. For example, pulse rate and/or pulse energy provided by the optical energy source can be at least partially controlled by the detected speed or changes in position. In this manner, a substantially uniform pattern or density of thermal damage can be generated over a region of skin, even if the optical arrangement is translated manually and translation speed/direction may not be exactly constant. Such sensor can also be used to provide appropriate pulse durations and intervals when the optical arrangement is translated over a particular region of skin a plurality of times. Imaging and/or positional sensors may also be provided to detect areas not to be treated (e.g. lips, eyes, pigmented areas, and the like). An optional control system and interface can also be provided so the user of the device can designate specific treatment areas (and/or areas not to be treated) via a graphical interface, and optionally to facilitate desired levels of re-treatment in regions that have already been treated.

In still further non-limiting examples, fractional damage to the skin can be generated using chromophores. In such non-limiting examples, a fractional pattern of chromophores can be applied to a region of skin tissue. After such application, the entire region can be exposed to light of appropriate wavelengths. Such light can be selectively absorbed by the locations containing the chromophore, generating thermal damage on the chromophore-treated spots and maintaining relatively undamaged healthy tissue in the skin areas between such spots. The general use of chromophores with optical energy to selectively absorb light in biological tissue is known in the art, and such systems may be used in non-limiting examples of the present disclosure.

In further non-limiting examples, patterns of ultrasound energy may be applied over regions of the body in a discontinuous or fractional pattern. Such application of ultrasound can generate small regions of thermally-damaged tissue surrounded by undamaged, healthy tissue.

In other non-limiting examples, fractional damage to the skin may be produced mechanically. For example, an array of needles can penetrate the skin repeatedly to generate small, separated wound regions surrounded by unaffected tissue. The needles can be either solid needles or hollow “coring” needles, where such coring needles may further remove small regions of skin tissue.

In still further non-limiting examples, fractional tissue damage can be generated in skin tissue using a single needle or an array of needles to which radiofrequency (“RF”) energy is applied. In such non-limiting examples, the needles can act as electrodes, and the RF energy delivered to the tissue adjacent to the needles can cause thermal damage in that local tissue. The general use of RF energy delivered by needles to generate damage in tissue is known in the art, and RF energy parameters needed to produce a desired amount of local tissue damage are well established.

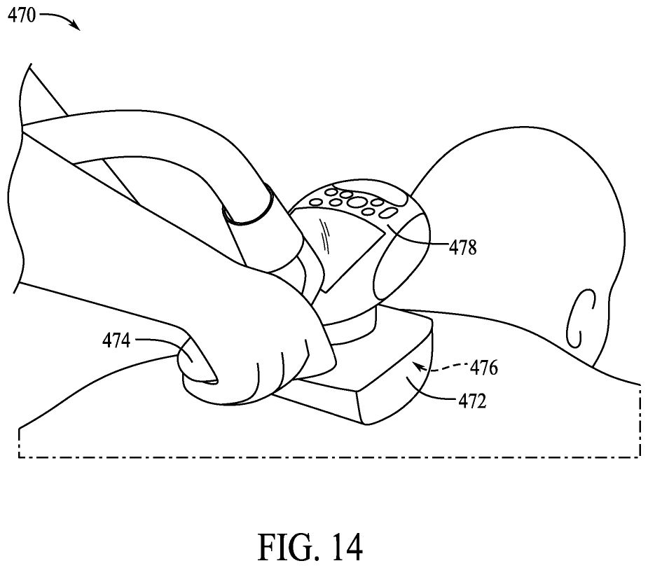

FIG. 14 shows a side view of a schematic illustration of a treatment system 470, which can be a specific implementation of the treatment system 100. Thus, the treatment system 470 pertains to the treatment system 100 (and vice versa). The treatment system 470 can also cover large swaths of a subject, which can be important for eliciting the desired metabolic response, or response associated with the metabolic function of the subject. The treatment system 470 can include a housing 472, a handle 474 coupled to the housing 472 (or a grip coupled to the housing 472), an energy source 476 coupled to the housing 474, and a user input device 478 coupled to the housing 472. In some non-limiting examples, with the handle 474, a user can move the energy source 476 to different locations on the subject to treat different regions of the skin tissue. Thus, the treatment system 470 can be handled so that a user can move the energy source 476 to different locations. In addition, when the treatment system 470 receives a user input from the user input device 478, the energy source 476 can deliver energy to create the plurality of treatment regions in the skin tissue.

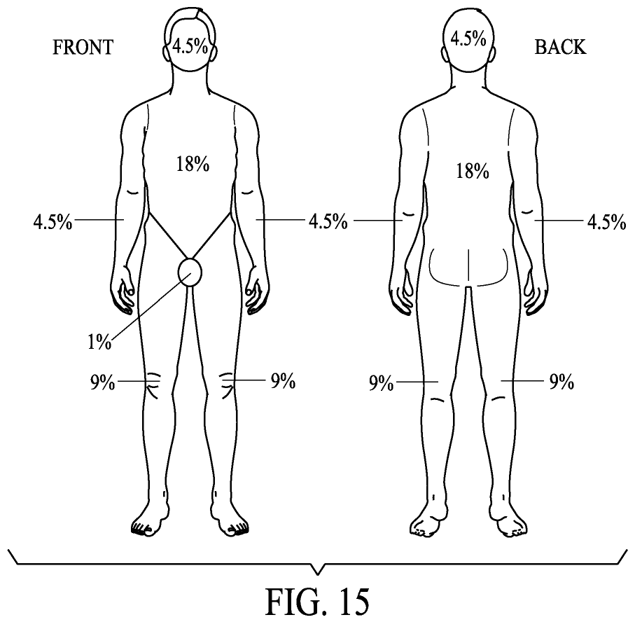

In some non-limiting examples, the percentage of total skin area covered by a treatment can be estimated using the so-called “Rule of Nines,” which is often used to estimate the amount of skin damage incurred by burn victims. The Rule of Nines is graphically illustrated in FIG. 15. For example, the entire head and neck area constitute about 9% of the body's total surface area. Other percentages are approximately: entire right or left arm—9% each; entire frontal (anterior) torso—18%; entire rear (posterior) torso—18%; entire left or right leg—18% each; groin area—1%.

Accordingly, based on these approximate percentages, it can be seen that a typical cosmetic fractional resurfacing treatment, which would at most cover the face and parts of the neck, would treat less than about 5% of the total skin area. In contrast, fractional damage treatments of greater than about 20% of the total skin area can be achieved, e.g., by treating the entire front torso, the entire back of the torso, both arms, a single leg, the front of both legs, or the back of both legs. Such treatment regions, which are much larger than those used in cosmetic treatments, are needed to generate a systemic response to the total tissue damage as described herein.

Thus, exemplary methods and devices are disclosed herein that can generate fractional patterns of mechanical damage, thermal damage, and/or ablation over large regions of a subject's skin, e.g., greater than about 20% of the total skin area. Depending on the specific modality used, such tissue damage may extend from the skin surface to within the skin tissue, or the damage may be substantially or entirely below the skin surface (e.g., when using ultrasound or non-ablative focused optical energy). Regions to be treated can be much larger than those treated in cosmetic fractional procedures, and can be performed over different parts of the body such as the torso and limbs.

Such large-scale fractional treatment may induce enhanced metabolism rates and lead to many desirable changes in the body such as, e.g., improved insulin resistance, improvements to metabolic syndrome conditions (e.g. reduced blood pressure, reduced high blood sugar, improved cholesterol and/or triglyceride levels), reduced waist circumference, improved cognitive function, etc. while avoiding the trauma and complications that generally result from severe burns. As a primary effect, such a hypermetabolic state can lead to steady weight loss without the need for intense exercise or diet regimens.

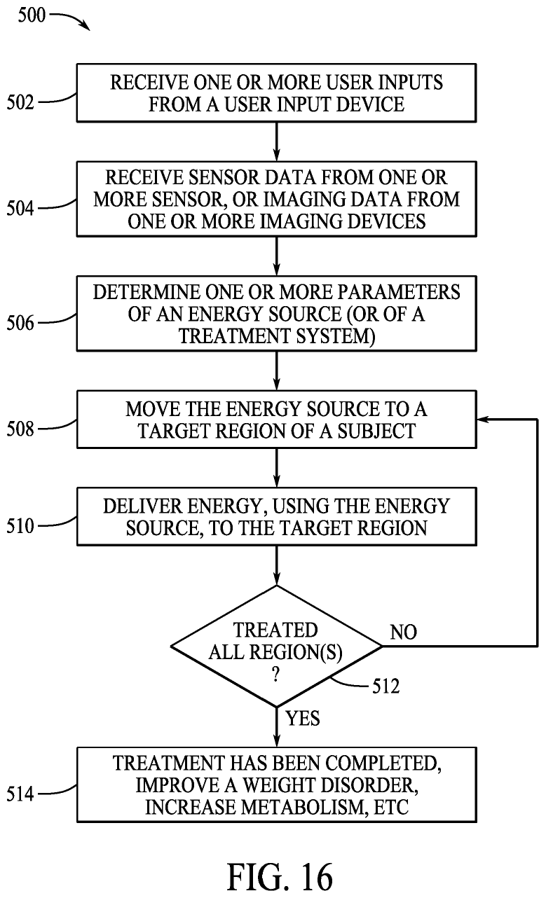

FIG. 16 shows a flowchart of a process 500 of at least one of increasing a metabolism of a subject, improving a weight disorder of the subject, improving one or more diseases associated with the weight disorder, decreasing a total amount of fat of the subject, decreasing a total weight of a subject, etc. The process 500 can be implemented using any of the treatment systems described herein as appropriate. In addition, the process 500 can be implemented using one or more computing devices, as appropriate.

At 502, the process 500 can include a computing device receiving one or more user inputs from a user input device (e.g., from a user that is to implement the treatment). In some cases, a user input can be indicative of one or more parameters of the subject, which can include a type of energy source to be used (e.g., an optical source, an ultrasound source, a RF source, a thermomechanical source, etc.), the number of target regions and a corresponding location of a respective target region, a density of the treatment regions (e.g., the total treatment surface of all treatment regions in a target region relative to the total surface area of the target region), the size of each treatment region (e.g., the width, the depth, etc.), the surface area of a target region, the surface are of all the target regions collectively (e.g., corresponding to the region of the BSA of the subject), the regions of the skin tissue of the subject to avoid (e.g., a sensitive region of the subject), etc.

At 504, the process an include a computing device receiving sensor data from one or more sensors, receiving imaging data from one or more imaging devices (or systems), etc. In some cases, this can include a distance, from a distance sensor, between an energy source and the skin tissue. In some cases, this can include imaging data, and the computing device can generate, using the imaging data, a 3D volume of the subject (e.g., to determine a surface area of the subject). In some configurations, this can include receiving an image from an image sensor (e.g., of a camera), and a computing device can determine a skin tone of the subject using the image, can determine a density of hair (e.g., at a desired target region), a coarseness of hair (e.g., at a desired target region), etc. In some non-limiting examples, this can include a computing device receiving imaging data (e.g., ultrasound imaging data), and determining a skin thickness using the imaging data.

At 506, the process 500 can include a computing device determining one or more parameters of the energy source (or a treatment system). In some cases, the one or more parameters can be determined based on one or more desired features including, for example, a type of energy source to be used (e.g., an optical source, an ultrasound source, a RF source, a thermomechanical source, etc.), the number of target regions and a corresponding location of a respective target region, a density of the treatment regions (e.g., the total treatment surface of all treatment regions in a target region relative to the total surface area of the target region), the size of each treatment region (e.g., the width, the depth, etc.), the surface area of a target region, the surface are of all the target regions collectively (e.g., corresponding to the region of the BSA of the subject), the regions of the skin tissue of the subject to avoid (e.g., a sensitive region of the subject), etc. For example, a computing device can receive the one or more desired features (e.g., as one or more corresponding inputs) and can determine the one or more parameters, based on the one or more desired features. In some cases, the one or more parameters can be the energy delivered by the energy source (e.g., the pulse width of a pulse of a laser, an amplitude of the pulse, etc.), the number of laser beams to be split from the laser beam (e.g., with each corresponding to a respective treatment region), the distance between the energy source and the skin tissue, the fluence of the laser, the beam width of each individual lasers, the duration of application of the energy (e.g., a laser beam), the Rayleigh range of the laser, the focused spot size, the wavelength of the energy (or electrical signal supplied to the energy source), the pattern of the energy to be delivered (e.g., by electrically exciting particular pins, electrodes, electrically heating particular pins, or blocking particular laser beams and allowing others to pass, etc.), the wavelength of the laser, etc.

In some non-limiting examples, a computing device can determine, using an image, one or more skin features of a subject, which can include a density of hair, a coarseness of hair, etc., and can determine the one or more parameters (e.g., the amount of energy delivered to the skin tissue) based on the one or more skin features. For example, denser amounts of hair and coarser hair can require more energy to create a respective treatment region and vice versa (e.g., because the hair, rather than the skin tissue absorbs some of the energy). In some cases, a computing device can determine a total surface area of the subject, and can determine a total desired treatment surface, using the total surface area of the subject. For example, the 3D volume of the subject can be used to determine the total surface area, or can use an equation (e.g., the Meeh equation), such as by receiving the weight of the subject and determining the total surface area using the weight of the subject. In some cases, the computing device can determine the total desired treatment surface, based on the determine total surface area (or received, such as from a user input), by, for example, multiplying the total surface area by a desired multiple (e.g., 20 percent, 30 percent, etc.). Then, a computing device can identify one or more target regions that satisfy the total desired treatment surface, using, for example, a user input indicative of the desired locations (e.g., a user can select the locations of the one or more target regions).

At 508, the process 500 can include a computing device moving the energy source to a target region of the skin tissue of the subject. In some cases, this can include a computing device causing a robot arm to move the energy source to, near, etc., a target region of the skin tissue of the subject.

At 510, the process 500 can include a computing device delivering energy, using the energy source, and according to the one or more parameters, to the target region to create a plurality of treatment regions in the target region of the skin tissue. In some cases, this can occur while the energy source is stationary (e.g., after having moved at the block 508), for example, the energy source delivers energy to simultaneously create all the plurality of treatment regions in the target region of the skin tissue. In other cases, the energy source 508 can be moved after a subset of the treatment regions in the target region have been created in the skin tissue. For example, a computing device can move the energy source to a first location, can cause the energy source to deliver first energy to create a first subset of the plurality of treatment regions in the target region of the skin tissue while the energy source is stationary (e.g., to create a first row of treatment regions), can move the energy source to a second location, can cause the energy source to deliver second energy to create a second subset of the plurality of treatment regions in the target region of the skin tissue while the energy source is stationary (e.g., to create a second row of treatment regions), and so on, until all the desired treatment regions have been created in the target region of the skin tissue.

At 512, the process 500 can include a computing device determining whether or not all the target region(s) have been treated. If at the block 512, the computing device determines that the all the target regions have been treated, the process 500 can proceed to the block 514. If, however, the computing device determines that all the target regions have not been treated, the process 500 can proceed back to the block 508 to move the energy source to another target region (e.g., a different treatment region) and subsequently deliver energy to the another target region.

At 514, the process 500 can include the treatment having been completed. In some non-limiting examples, the process 500 can include increasing a metabolism of a subject (e.g., increasing a basal metabolic rate of the subject, increase a metabolism of a skin tissue of a subject) improving a weight disorder of the subject (e.g., decreasing a total amount of fat of the subject, decreasing fat in one or more regions of the subject including a subcutaneous region, decreasing a thickness in fat of one or more regions of the subject including a subcutaneous region, etc.), improving one or more diseases caused by the weight disorder (e.g., decreasing insulin resistance, reducing blood pressure, reducing blood sugar, decreasing cholesterol levels, decreasing triglyceride levels, improving heart diseases, improving a mental illness disorder, decreasing pain (e.g., in one or more joints of the subject), etc.

In some non-limiting examples, the process 500 can be repeated after a number of days (e.g., one, two, three, four, five, six, seven, etc.). For example, after a number of days (e.g., 3 days, 7 days, etc.) the treatment regions have healed and the transitory increase in metabolic rate (e.g., basal metabolic rate) has subsided (e.g., because the treatment regions have healed). In other non-limiting examples, the process 500 can be repeated after a number of hours (e.g., 10 hours, 12 hours, etc.), after which point the increase in metabolic rate peaks and begins to decrease. For example, a subsequent iteration of the process 500 can target different target regions. In other words, all of the target regions from the first iteration of the process 500 can be different than all of the target regions form the second iteration of the process 500. In this way, the same target regions are not targeted multiple times too soon, which could prevent adequate healing of the treatment regions within the target region.

### EXAMPLES

The following examples have been presented in order to further illustrate aspects of the disclosure, and are not meant to limit the scope of the disclosure in any way. The examples below are intended to be examples of the present disclosure and these (and other aspects of the disclosure) are not to be bounded by theory.

### Example 1

Exposure of large areas (e.g., greater than or equal to 30% of the total body surface area of a subject) of skin to fractional laser leads to an increase in metabolism and weight loss. Devices to achieve large area exposure include lasers integrated into beds (e.g. similar to tanning beds), devices that surround the limbs, devises that surround the torso, etc. The devices can be built as various laser delivery systems including but not limited to scanning lasers where the laser source is physically moved (e.g., robotically) around the body or multiplexed fiber lasers with multiple smaller scanning patterns or hundreds of fibers, each of which provides a single laser beam for exposure and the laser source alternates which fiber is being used to deliver energy. It has been recognized that weight loss that severe burn patients experience during recovery may be due to the increase in metabolism caused by wound healing. Fractional lasers make tiny micro-wounds and the non-limiting examples herein can create a modulated wound healing environment to achieve an increase in overall metabolic rate without causing the extensive tissue damage and negative systemic response seen in severe burn cases. Exposing a large area of skin to fractional treatment to produce an effect on metabolism and the devices designed to do so are not believed to have been created. For example, conventional fractional treatments are generally applied to substantially small areas of the body including the face, with the scarred areas comprising 10% or less of the total skin area being exposed to fractional laser treatment. The research herein has shown that exposing substantially 30% or more of skin to fractional laser can lead to an increase in metabolism as evidenced by UCP-1 signals in fat, increases in noradrenaline levels, and can result in weight loss (e.g., without targeting lean mass, including muscle mass). The method of causing weight loss, modulating metabolism, etc., by exposing greater than 30% of the skin to fractional laser is not believed to have been previously shown, implemented, etc. Devices that can achieve this large area exposure of skin can include robotic lasers that can move around the body to impart a fractional pattern of laser exposure, tanning bed-like devices that include integrated lasers, which can include but are not limited to fiber lasers, that move around inside the device to apply fractional laser to large areas of skin.

A new study was designed for weight loss in mice. A first aim was to demonstrate weight loss in mice with large area fractional laser therapy, and second aim was to explore the dosimetry to evaluate extent of the effect. The mouse model C57BL/6 mice fed on a high-fat diet for 4 weeks to prepare overweight mice for study. The following were different parameters tested including body area coverage, laser density, and ablative vs. non-ablative for 8 control mice.

### Example 2

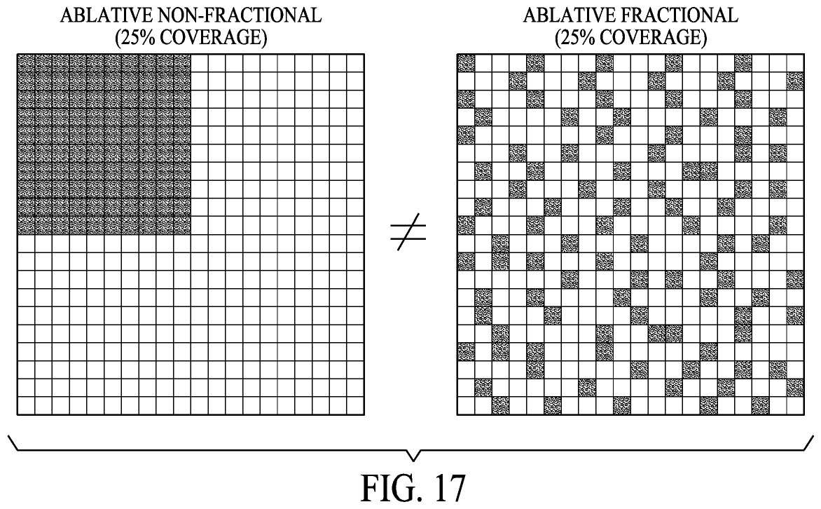

FIG. 17 shows a graphical representation to illustrate the concept of confluent laser treatments and fractional laser treatments. The darker zones indicate the thermally damaged areas. Although in both cases, the same total area is covered by the laser treatment (i.e., 25%), the treatment outcome is expected to be markedly different. Fractional photothermolysis (right) with the same laser settings, but using a pattern leaving intervening unaffected tissue in between reduces side-effects and induces wound healing without formation of scarring and fibrosis.

Mice were treated in the following example. For example, 22-week-old male C57BL/6J mice (N=5+3) were used. A CO₂ laser was used deliver ablated fractional photothermolysis (“aFP”) on the back of each mouse, covering approximately 30% of the body surface area of each mouse, with the energy delivered to each treatment region being 20 mJ, and the density of the treatment region being 10%. There was a 5-day monitoring period, and the Δ weight (i.e., change in weight) was analyzed, the IL-6 markers were analyzed, the Catecholamine levels were analyzed, and the liver of each mouse was assessed.

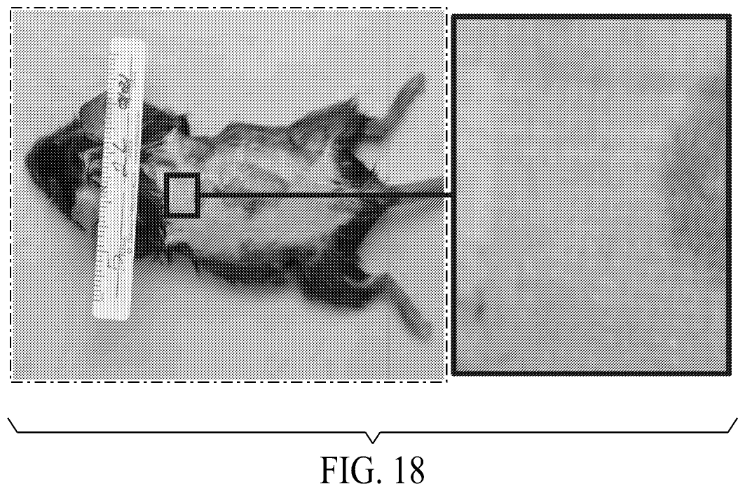

FIG. 18 shows a photograph of a mouse of the experimental setup.

In this example, 16, 22-week-old male C57BL/6J mice (N=8+8) were treated according to their specific group A CO₂ laser was used for aFP exposure on the back of each mouse, covering approximately 30% BSA (E=17 mJ, 10%). An EchoMRI (Houston, TX) was used for NMR-assisted body composition analysis. There was a 6-day monitoring period using a Promethion High-Definition multiplexed respirometry system for rodent analysis (available at Sable Systems, Las Vegas, NV). The metabolic quantification was conducting using the Prometheon (Sable Systems).

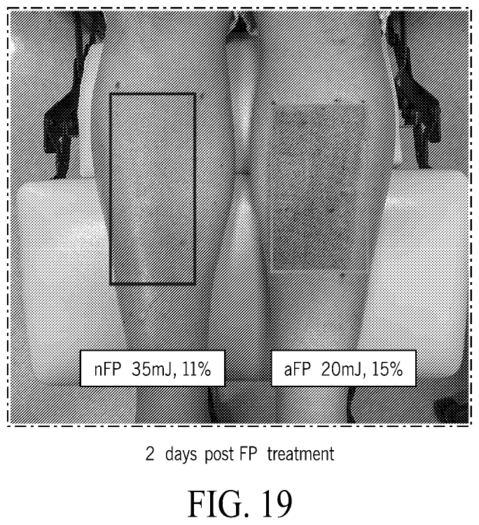

FIG. 19 shows a photograph of a non-ablation FP (“nFP”) on one leg of a subject with 35 mJ per treatment region and a density of 11% (of treatment regions), and an ablation FP (“aFP”) on the other leg of the subject with 20 mJ per treatment region and a density of 15% (of treatment regions). The photograph was taken 2 days after the treatment.

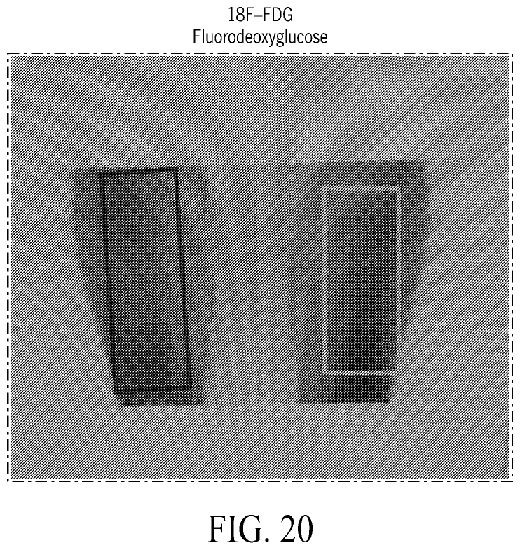

FIG. 20 shows a positron emission tomography (“PET”) image of both legs of the subject of FIG. 19.

As shown in this example, the nFP and aFP can enhance Baseline Metabolic Rate (“BMR”) of the skin, and the FP allows the modification of the baseline metabolic rate of skin. The large area FP can have potential to become an adjuvant therapy for weight management.

### Example 2

The following example shows the effects of large area fractional phototherymolysis treatment on mouse metabolism.

In this example, 22-week-old male C57Bl/6J mice were used. The 8 mice were exposed to fractional laser treatment over a large body surface area (˜30%). The density of the treatment regions was 10% density and 17 mJ were delivered per pulse (e.g., with each individual laser beam being 17 mJ to create a respective treatment region). There were 8 mice that were used as untreated mice. The results from this example show an increase in metabolism, which was measured using the Promethion system

As shown from the figures in the example, there is a clear differentiation in energy utilization from 12 h to ˜72 h after the laser treatment that normalizes over 4-7 days without significant changes in body mass or body composition

### Example 3

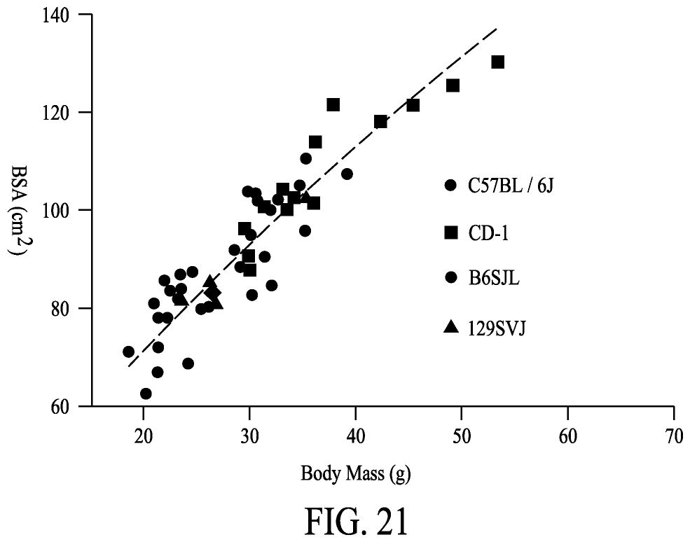

FIG. 21 shows a graph of body mass versus body surface area (“BSA”) versus body mass with a fitted function (e.g., using the Meeh equation). The Meeh equation is the following:

BSA=k*mass^{0.667} (1)

In the Meeh equation k is determined empirically for each species, which is k=10 for BL6 mice. Thus, 30 g mice have a BSA of 95 cm². The treatment area was determined to be 20 cm² at a 10% density (e.g., where 20 cm² of 95 cm² is a treatment area of 21%). At 10% density, the Absolute Treaded Surface Area (“ATSA”) of 2.1%.

Experiment Schedule

| | | Treated | | | | Absolute |
|---|---|---|---|---|---|---|
| | | Body | | | | Treated |
| | Number | Surface | Laser | Laser | Laser | Surface |
| Experiment | of Mice | Area | Density | Energy | Type | Area |
| 1 | 8 | — | — | — | — | — |
| 1 | 8 | 20% | 10% | 17 mJ | Ablative | 2.2% |
| 2 | 4 | 5% | 10% | 17 mJ | Ablative | 0.6% |
| 2 | 4 | 10% | 10% | 17 mJ | Ablative | 1.1% |
| 2 | 4 | 32% | 15% | 17 mJ | Ablative | 5.3% |
| 2 | 4 | 20% | 10% | 9 mJ | Non- | 2.0% |
| | | | | | Ablative | |
| 3 | 4 | 15% | 10% | 17 mJ | Ablative | 1.7% |
| 3 | 4 | 10% | 10% | 9 mJ | Non- | 1.0% |
| | | | | | Ablative | |
| 3 | 4 | 25% | 15% | 9 mJ | Non- | 3.6% |
| | | | | | Ablative | |
| 3 | 4 | 32% | 20% | 9 mJ | Non- | 6.3% |
| | | | | | Ablative | |

Table 2 shows the experiment schedule for various experiments, the results of which are shown in the following figures.

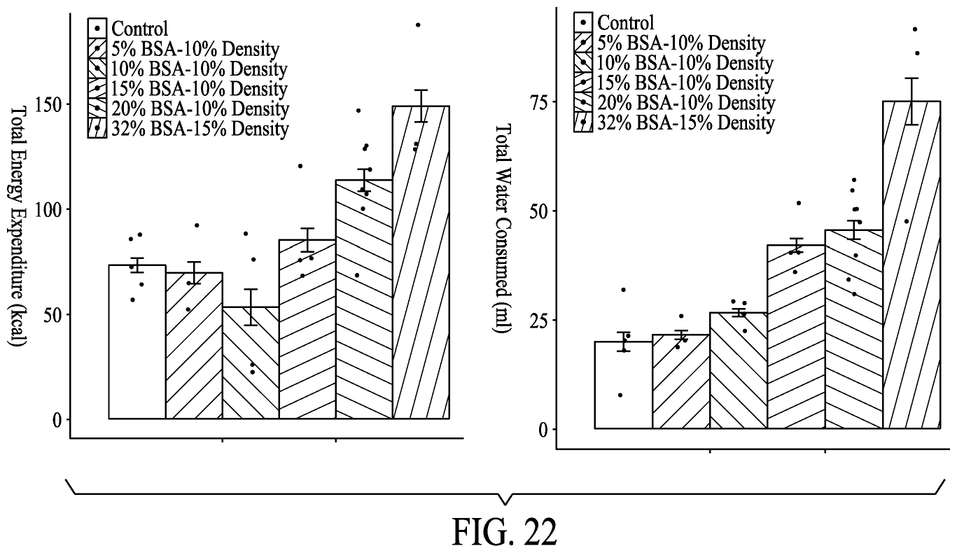

FIG. 22 shows a graph of total energy expenditure for six groups, and a graph of total water consumption for the six groups.

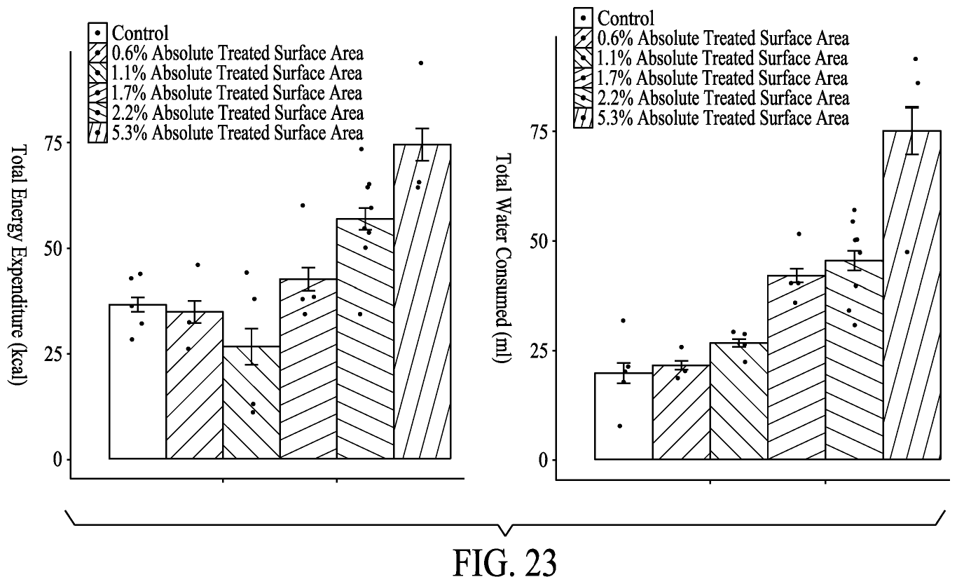

FIG. 23 shows a graph of total energy expenditure for six groups, and a graph of total water consumption for the six groups.

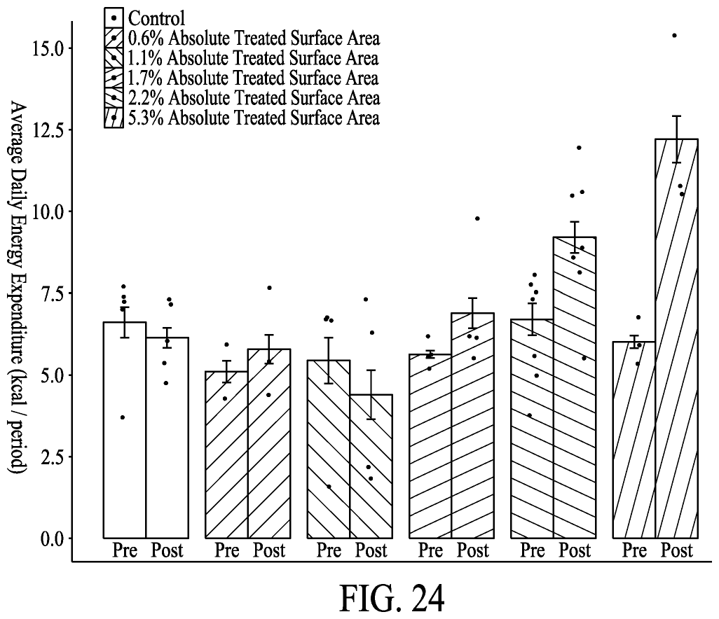

FIG. 24 shows a graph of the average daily energy expenditure for six groups before and after treatment.

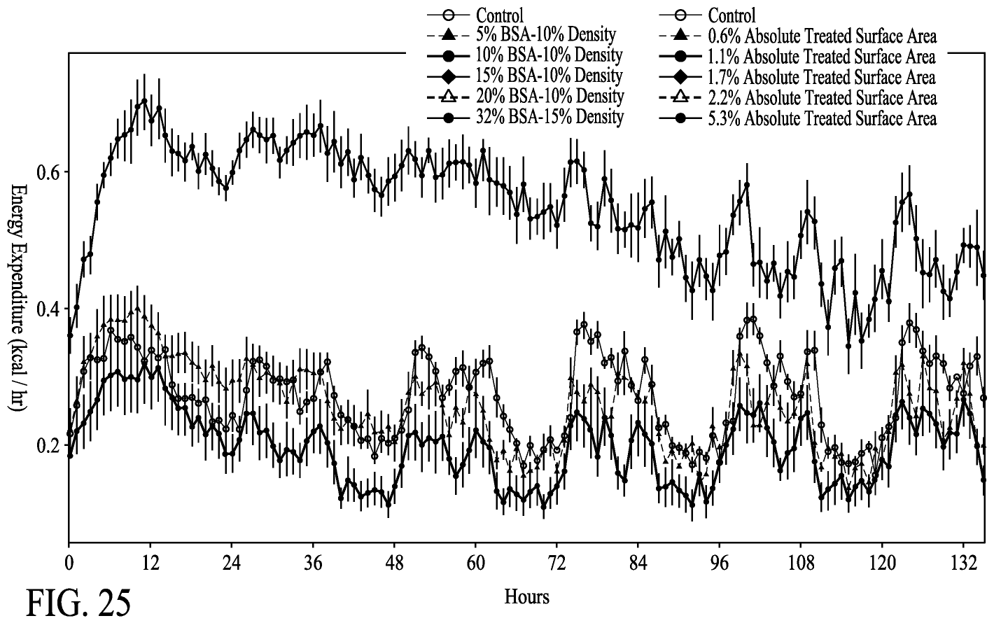

FIG. 25 shows a graph of the energy expenditure over time for six groups.

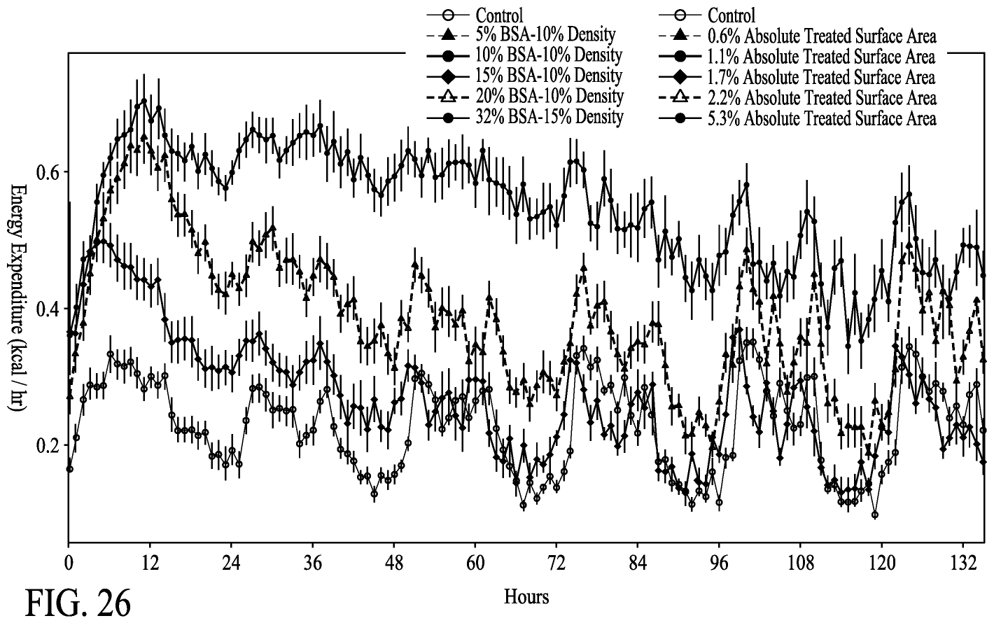

FIG. 26 shows a graph of the energy expenditure over time for six groups.

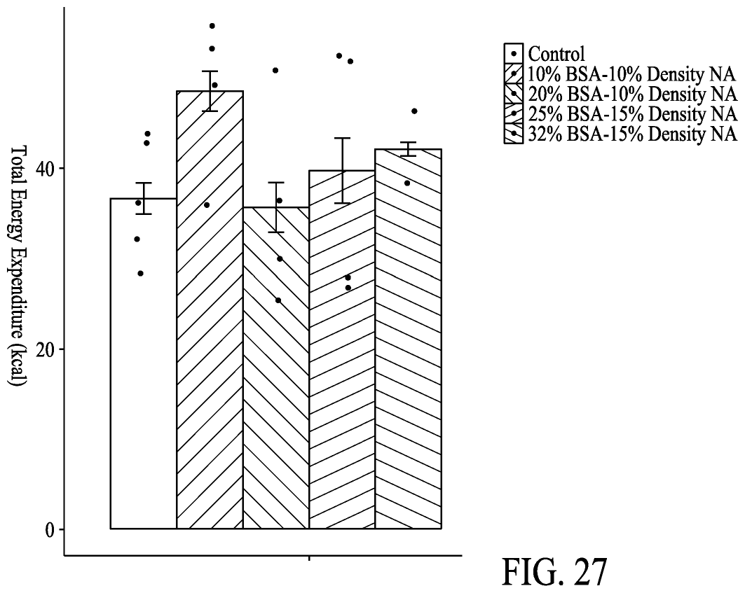

FIG. 27 shows a bar graph of the total energy expenditure for six groups.

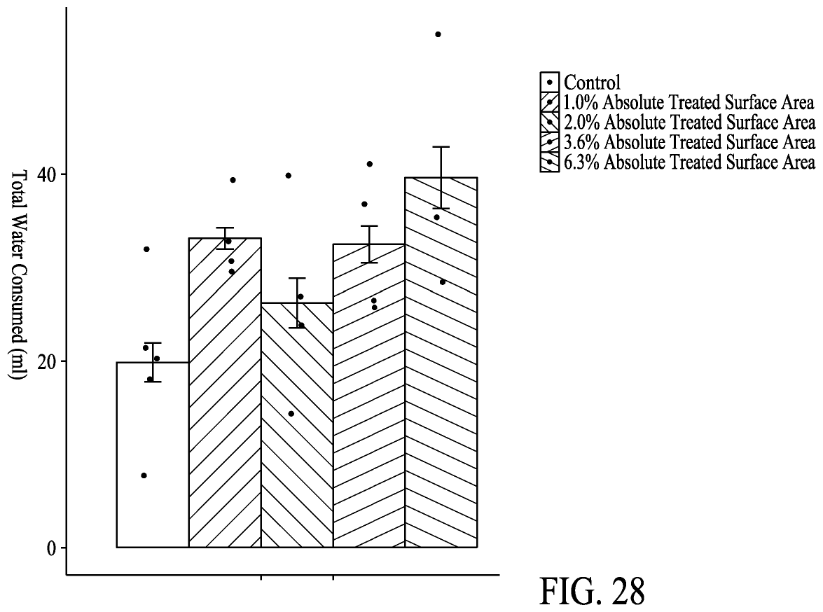

FIG. 28 shows a bar graph of the total energy expenditure for six groups.

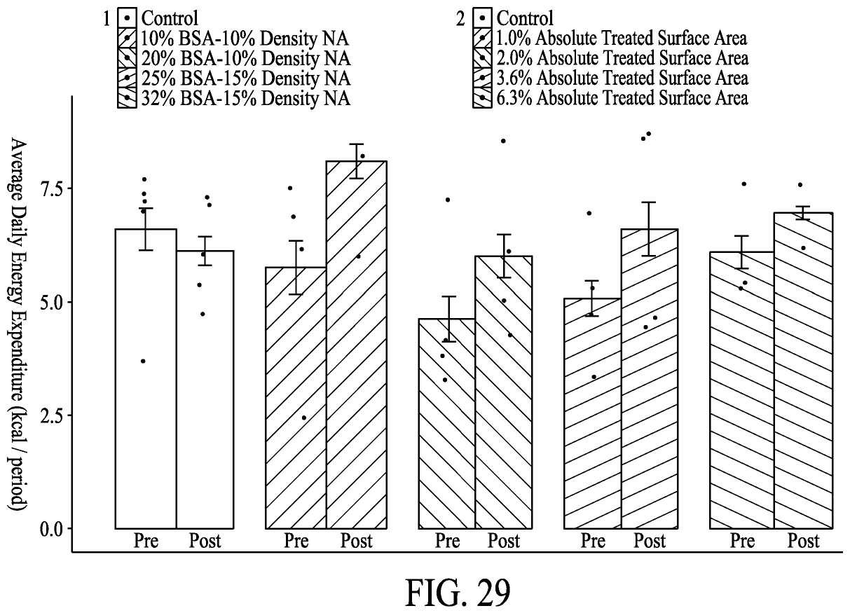

FIG. 29 shows a bar graph of the average daily energy expenditure for a first set of six groups, and a second set of six groups. The bars on the left side correspond to the first set of six groups (denoted “1”), and the bars on the right side correspond to the second set of six groups (denoted “2”).

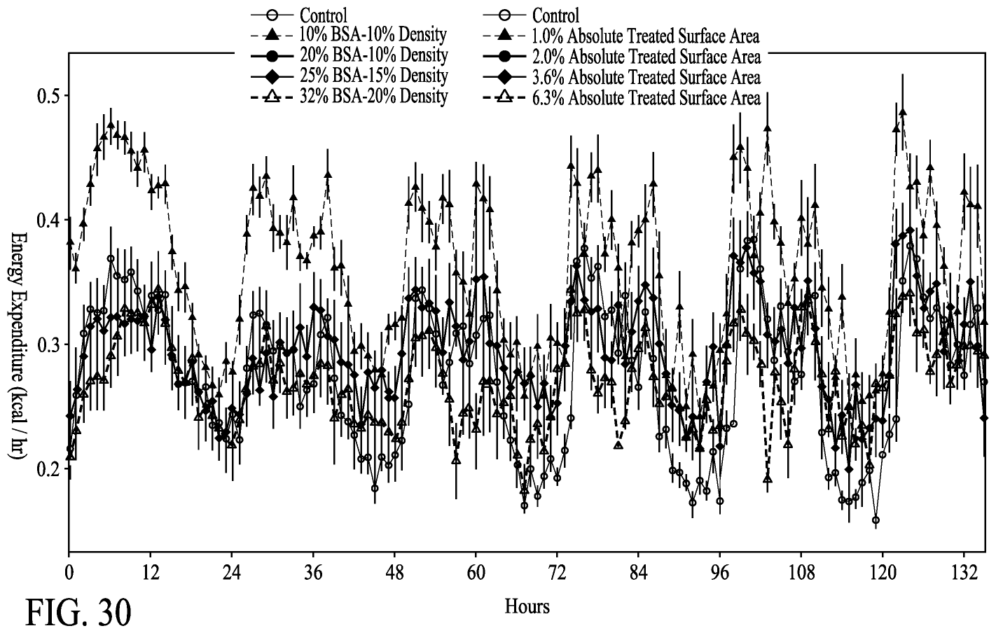

FIG. 30 shows a graph of the energy expenditure over time for six groups.

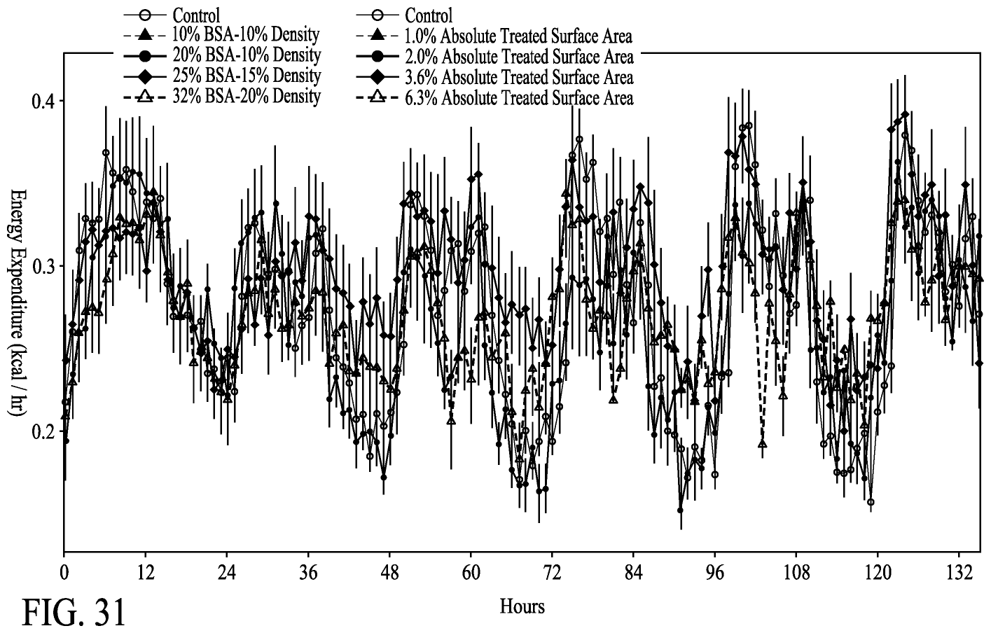

FIG. 31 shows a graph of the energy expenditure over time for six groups.

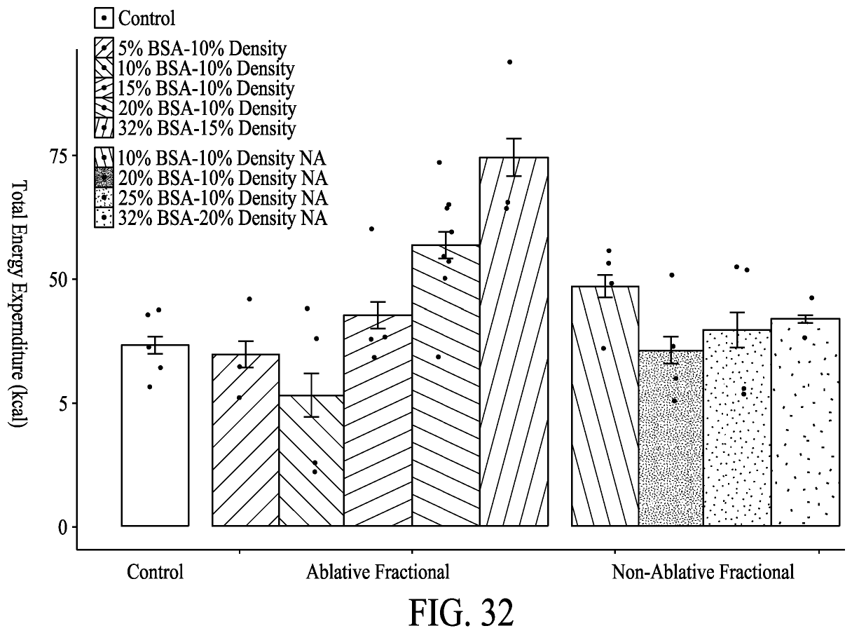

FIG. 32 shows a bar graph of the total energy expenditure for ten groups.

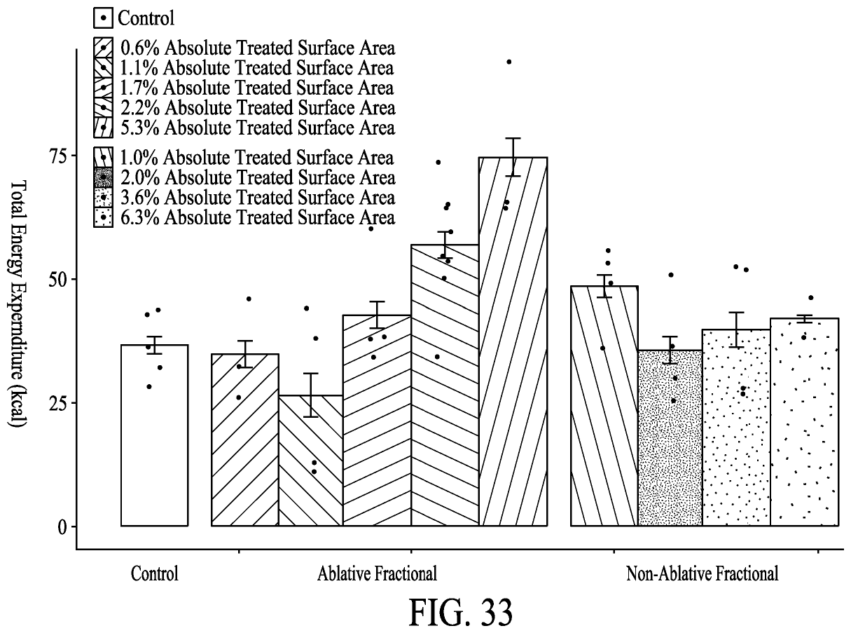

FIG. 33 shows a bar graph of the total energy expenditure for ten groups.

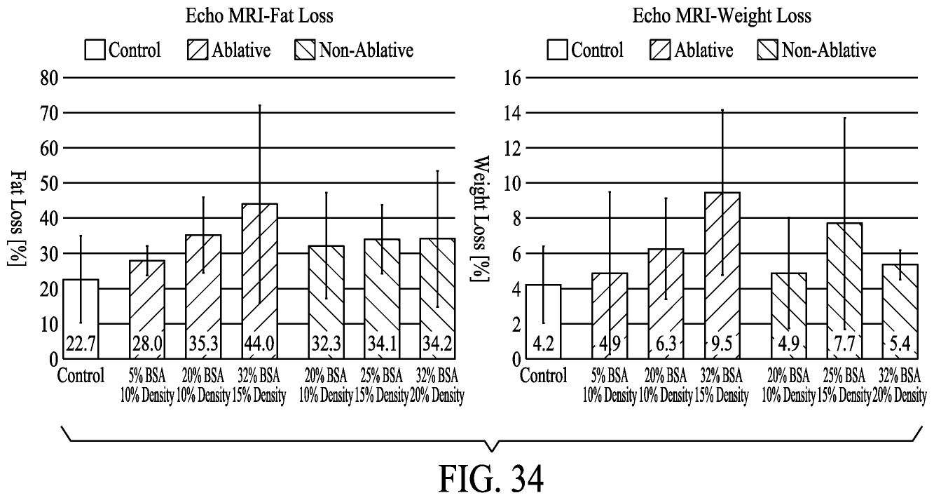

FIG. 34 shows a bar graph of the fat loss using EchoMRI for seven groups, and a bar graph of the weight loss using EchoMRI for the seven groups.

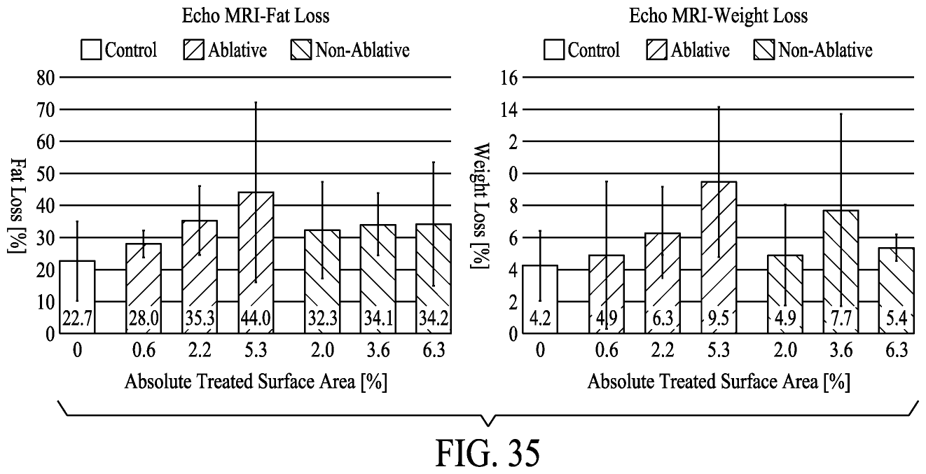

FIG. 35 shows a bar graph of the fat loss using EchoMRI for seven groups, and a bar graph of the weight loss using EchoMRI for the seven groups.

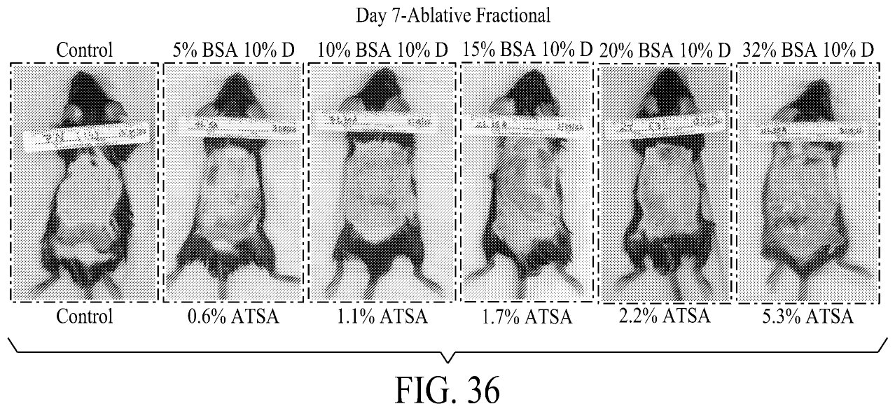

FIG. 36 shows photographs of mice from the ablative FP group.

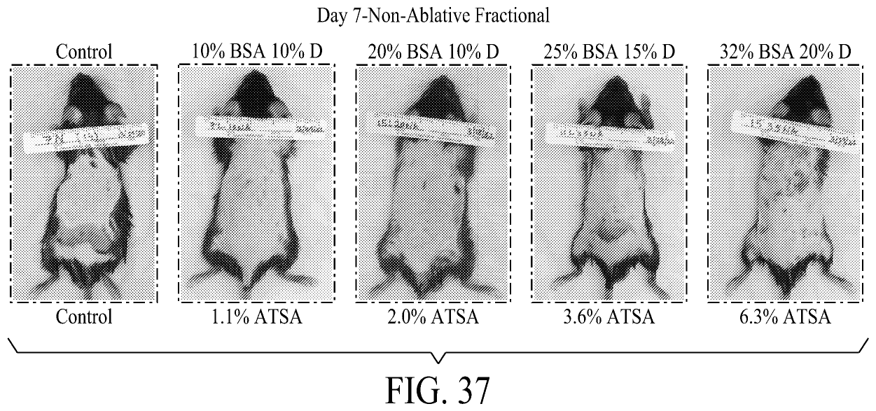

FIG. 37 shows photographs of mice from the non-ablative FP group.

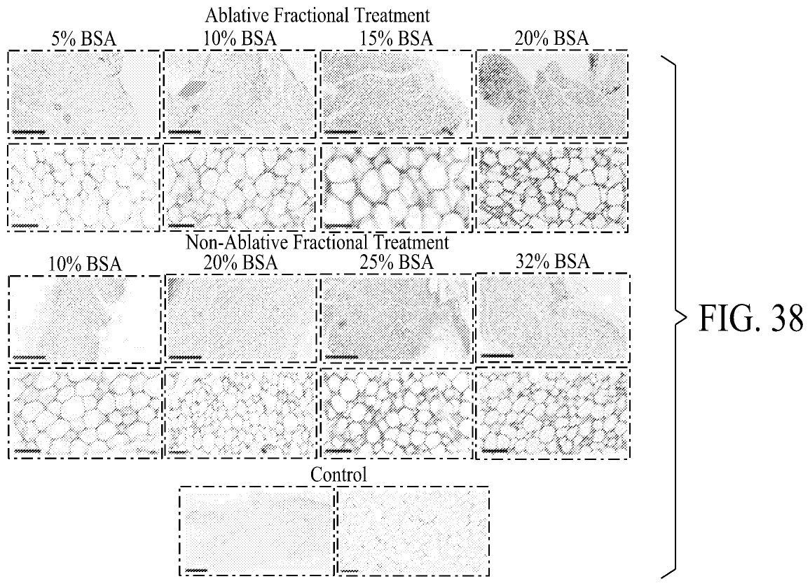

FIG. 38 shows images of white adipose tissue for different treatment groups.

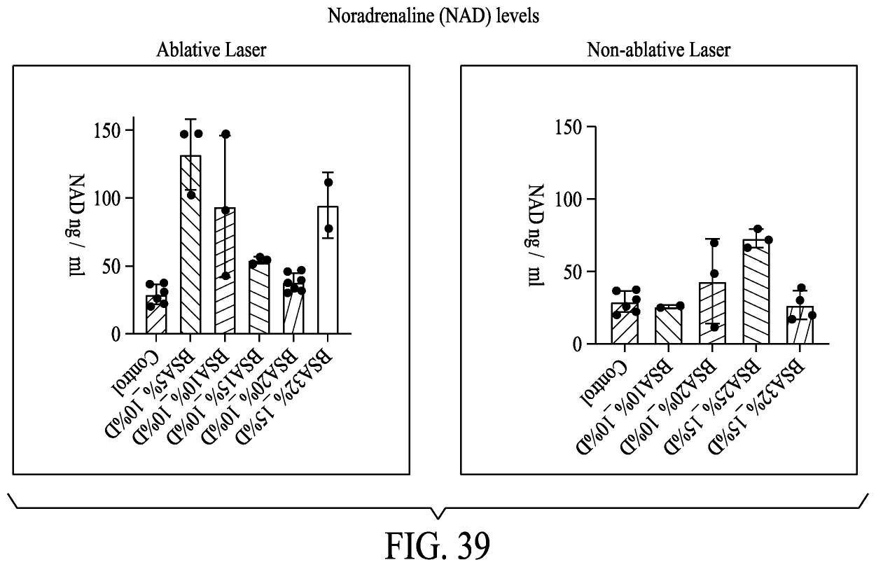

FIG. 39 shows a graph of the noradrenaline concentration for the ablative laser groups, and a graph of the noradrenaline concentration for the non-ablative laser group.

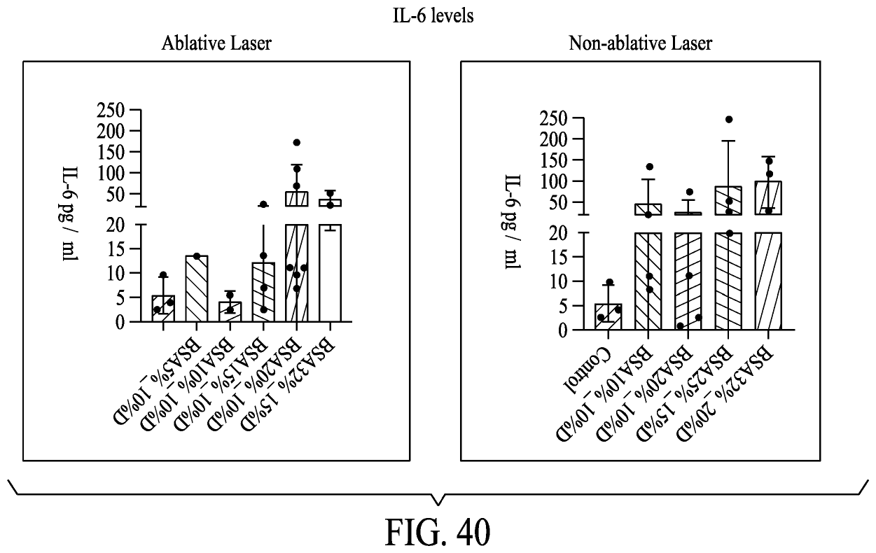

FIG. 40 shows a graph of the IL-6 concentration for the ablative laser groups, and a graph of the IL-6 concentration for the non-ablative laser group.

Injury of the skin caused by burns, incisions, or blunt forces, induces an immune response. These immune responses seem to be specific to the etiology of the injury. Skin trauma causes an inflammatory response that includes the upregulation of inflammatory cytokines such as IL-6. IL-6 has been detected at the wound sites and in the blood of burned mice and has been linked to positive healing response, including enhancing collagen deposition, granulation tissue formation, and neo-vascularization. In addition to wound healing functions after burn trauma, IL-6 mediates the browning of white adipose tissue (WAT) and hypermetabolism. High quantities of mitochondria and the expression of the browning marker uncoupling protein 1 (UCP1) distinguish brown adipocyte tissue (BAT) from WAT.

Because fractional laser creates an array of microscopic treatment zones (MTZ) of thermal injury, this kind of thermal injury was assessed to see if it could increase levels of IL-6. It was found that elevated levels of IL-6 in the serum of all mice treated with laser compared to sham control mice. Browning of adipocytes was determined by immunohistochemistry (IHC) of Ucp1. It was found that elevated Ucp1 expression in all laser-treated groups compared to sham after one laser treatment on day seven. These results link the browning of adipocytes to high levels of IL-6 after fractional treatments of a large area in mice.

Prolonged adrenergic stress, measured by noradrenaline elevation, follows a burn injury. The systemic elevation of catecholamines leads to the activation of the beta3-adrenergic receptor and the induction of browning of WAT by the expression of Ucp1, and consequently the increase in the rate of lipolysis. Noradrenaline levels were measured to determine if laser treatment of a large area of mice triggers an adrenergic response. Levels of noradrenaline increased in laser-treated mice, particularly in the ablative laser type, from all selected BSA. Levels of noradrenaline in non-ablative laser-treated mice were slightly elevated in mice treated at 20 and 25% BSA.

As shown, there is a loss in fat mass and overall weight as well as upregulation of biomarkers such as UCP1, Noradrenalin, and IL-6. Also there was an increase of mechanistic effects such as browning of adipocytes, inflammation, and wound healing activity.

The present disclosure has described one or more preferred non-limiting examples, and it should be appreciated that many equivalents, alternatives, variations, and modifications, aside from those expressly stated, are possible and within the scope of the invention.

It is to be understood that the disclosure is not limited in its application to the details of construction and the arrangement of components set forth in the accompanying description or illustrated in the accompanying drawings. The disclosure is capable of other non-limiting examples and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, unless otherwise limited or defined, discussion of particular directions is provided by example only, with regard to particular non-limiting examples or relevant illustrations. For example, discussion of “top,” “front,” or “back” features is generally intended as a description only of the orientation of such features relative to a reference frame of a particular example or illustration. Correspondingly, for example, a “top” feature may sometimes be disposed below a “bottom” feature (and so on), in some arrangements or non-limiting examples. Further, references to particular rotational or other movements (e.g., counterclockwise rotation) is generally intended as a description only of movement relative a reference frame of a particular example of illustration.

In some non-limiting examples, aspects of the disclosure, including computerized implementations of methods according to the disclosure, can be implemented as a system, method, apparatus, or article of manufacture using standard programming or engineering techniques to produce software, firmware, hardware, or any combination thereof to control a processor device (e.g., a serial or parallel general purpose or specialized processor chip, a single- or multi-core chip, a microprocessor, a field programmable gate array, any variety of combinations of a control unit, arithmetic logic unit, and processor register, and so on), a computer (e.g., a processor device operatively coupled to a memory), or another electronically operated controller to implement aspects detailed herein. Accordingly, for example, non-limiting examples of the disclosure can be implemented as a set of instructions, tangibly embodied on a non-transitory computer-readable media, such that a processor device can implement the instructions based upon reading the instructions from the computer-readable media. Some non-limiting examples of the disclosure can include (or utilize) a control device such as an automation device, a special purpose or general purpose computer including various computer hardware, software, firmware, and so on, consistent with the discussion below. As specific examples, a control device can include a processor, a microcontroller, a field-programmable gate array, a programmable logic controller, logic gates etc., and other typical components that are known in the art for implementation of appropriate functionality (e.g., memory, communication systems, power sources, user interfaces and other inputs, etc.).

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally it should be appreciated that a carrier wave can be employed to carry computer-readable electronic data such as those used in transmitting and receiving electronic mail or in accessing a network such as the Internet or a local area network (LAN). Those skilled in the art will recognize that many modifications may be made to these configurations without departing from the scope or spirit of the claimed subject matter.

Certain operations of methods according to the disclosure, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular non-limiting examples of the disclosure. Further, in some non-limiting examples, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as non-limiting examples of the disclosure, of the utilized features and implemented capabilities of such device or system.

As used herein, unless otherwise defined or limited, ordinal numbers are used herein for convenience of reference based generally on the order in which particular components are presented for the relevant part of the disclosure. In this regard, for example, designations such as “first,” “second,” etc., generally indicate only the order in which the relevant component is introduced for discussion and generally do not indicate or require a particular spatial arrangement, functional or structural primacy or order.

As used herein, unless otherwise defined or limited, directional terms are used for convenience of reference for discussion of particular figures or examples. For example, references to downward (or other) directions or top (or other) positions may be used to discuss aspects of a particular example or figure, but do not necessarily require similar orientation or geometry in all installations or configurations.

This discussion is presented to enable a person skilled in the art to make and use non-limiting examples of the disclosure. Various modifications to the illustrated examples will be readily apparent to those skilled in the art, and the generic principles herein can be applied to other examples and applications without departing from the principles disclosed herein. Thus, non-limiting examples of the disclosure are not intended to be limited to non-limiting examples shown, but are to be accorded the widest scope consistent with the principles and features disclosed herein and the claims below. The accompanying detailed description is to be read with reference to the figures, in which like elements in different figures have like reference numerals. The figures, which are not necessarily to scale, depict selected examples and are not intended to limit the scope of the disclosure. Skilled artisans will recognize the examples provided herein have many useful alternatives and fall within the scope of the disclosure.

Also as used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as “either,” “one of,” “only one of,” or “exactly one of.” Further, a list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more of each of A, B, and C. Similarly, a list preceded by “a plurality of” (and variations thereon) and including “or” to separate listed elements indicates options of multiple instances of any or all of the listed elements. For example, the phrases “a plurality of A, B, or C” and “two or more of A, B, or C” indicate options of: A and B; B and C; A and C; and A, B, and C. In general, the term “or” as used herein only indicates exclusive alternatives (e.g. “one or the other but not both”) when preceded by terms of exclusivity, such as “either,” “one of,” “only one of,” or “exactly one of.”

Various features and advantages of the disclosure are set forth in the following claims.

What is claimed is:

1. A treatment system comprising:
an energy source configured to thermally damage skin tissue of a subject;
a user input device configured to receive a user input;
a computing device configured to:
receive, from the user input device, the user input indicative of one or more operational parameters of the energy source;
based on the user input, control the energy source according to the one or more operational parameters to cause the energy source to thermally damage the skin tissue by creating a plurality of treatment regions within a target region of the skin tissue to increase a basal metabolic rate of the subject, wherein the target region includes a non-treatment portion that is interspersed among the treatment regions.

2. The treatment system of claim 1, wherein the computing device is further configured to control the energy source according to the one or more operational parameters to randomly distribute the treatment regions among the target region of the skin tissue.

3. The treatment system of claim 1, wherein the creation of the treatment regions are configured to decrease an amount of fat of the subject.

4. The treatment system of claim 1, wherein the plurality of treatment regions form an array of treatment regions within the target region;
wherein the array includes multiple columns and multiple rows; and
wherein the treatment regions are in the multiple columns and the multiple rows of the array.

5. The treatment system of claim 4, wherein the energy source is the transducer that is a light source; and
wherein the computing device is further configured to cause the light source to emit light towards the target region of the skin tissue to form the plurality of treatment regions of the skin tissue.

6. The treatment system of claim 5, wherein the computing device is further configured to cause the light source to emit a plurality of beams of light towards the target region of the skin tissue; and
wherein each beam of the plurality of beams creates a respective treatment region of the plurality of treatment regions of the skin tissue.

7. The treatment system of claim 5, wherein the light source is configured to generate a fractional illumination pattern that is directed at the target region to form the plurality of treatment regions and the non-treatment region.

8. The treatment system of claim 5, wherein each of the plurality of treatment regions of the skin is a non-ablative treatment region.

9. The treatment system of claim 8, wherein the controller is further configured to control the light source to deliver less than or equal to 9 mJ of energy to create each of the plurality of treatment regions.

10. The treatment system of claim 5, wherein each of the plurality of treatment regions of the skin is an ablative treatment region.

11. The treatment system of claim 10, wherein the controller is further configured to control the light source to deliver less than or equal to 17 mJ of energy to create each of the plurality of treatment regions.

12. The treatment system of claim 1, wherein the target region is at least one of:
10 percent of the total body surface area of the skin tissue of the subject;
20 percent of the total body surface area of the skin tissue of the subject;
30 percent of the total body surface area of the skin tissue of the subject; or
32 percent of the total body surface area of the skin tissue of the subject.

13. The treatment system of claim 12, wherein the target region does not include at least one of the genitals of the subject, or the head of the subject.

14. The treatment system of claim 1, wherein each of the plurality of treatment regions defines a treatment surface within the target region, and the non-treatment region defines a non-treatment surface within the target region;
wherein all the treatment surfaces of the plurality of treatment regions defines a total treatment surface area of the target region; and
wherein the percentage of the treatment surface area to total surface area of the target region is at greater than or equal to 10 percent.

15. The treatment system of claim 13, wherein the percentage of the treatment surface area to the total surface area of the treatment region is at least one of greater than or equal to 15 percent, 20 percent, 30 percent, or 32 percent.

16. The treatment system of claim 1, wherein each of the plurality of treatment regions defines a treatment surface, and the non-treatment region defines a non-treatment surface;
wherein all the treatment surfaces of the plurality of treatment regions defines a total treatment surface area of the target region; and
wherein the percentage of the treatment surface area to the total body surface of the subject is at least 1 percent.

17. The treatment system of claim 16, wherein the percentage of the treatment surface area to the total body surface of the subject is at least one of 2 percent, 3.6 percent, or 6.3 percent.

18. The treatment system of claim 1, wherein the formation of the plurality of treatment regions of the target region of the skin tissue decreases an amount of fat tissue at the target region.

19. The treatment system of claim 1, wherein the formation of the plurality of treatment regions of the target region of the skin tissue decreases a total amount of fat of the subject.

20. The treatment system of claim 1, wherein the formation of the plurality of treatment regions of the target region of the skin tissue transforms a fat cell that is white fat cell or beige fat cell into a brown fat cell.

21. The treatment system of claim 1, wherein the formation of the plurality of treatment regions of the target region of the skin tissue increases the amount of noradrenaline circulating through the bloodstream of the subject.

22. The treatment system of claim 1, wherein the energy source includes a transducer.

23. The treatment system of claim 1, wherein the energy source includes an electrical generator and one or more electrodes, the electrical generator being configured to direct an electrical signal to the one or more electrodes thereby thermally damaging the skin tissue.

24. The treatment system of claim 1, wherein the energy source is configured to create the plurality of treatment regions without creating an incision or a puncture at the target region of the skin tissue.

25. The treatment system of claim 1, wherein a treatment region of the plurality of treatment regions has a width of less than or equal to 1 millimeter.

26. The treatment system of claim 1, wherein a treatment region of the plurality of treatment regions does not extend into the subcutaneous tissue of the treatment region.

27. A treatment system comprising:
an energy source configured to thermally damage skin tissue of a subject;
a user input device configured to receive a user input;
a computing device configured to:
receive, from the user input device, the user input indicative of one or more operational parameters of the energy source;
based on the user input, control the energy source according to the one or more operational parameters to cause the energy source to thermally damage the skin tissue by creating a plurality of treatment regions within a target region of the skin tissue to decrease a total amount of fat of the subject, wherein the target region includes a non-treatment portion that is interspersed among the treatment regions.

28. A treatment system comprising:
an energy source configured to thermally damage skin tissue of a subject;
a user input device configured to receive a user input;
a computing device configured to:
receive, from the user input device, the user input indicative of one or more operational parameters of the energy source;
based on the user input, control the energy source according to the one or more operational parameters to cause the energy source to thermally damage the skin tissue by creating a plurality of treatment regions within a target region of the skin tissue to transform one or more white fat cells into one or more beige fat cells or one or more brown fat cells, wherein the target region includes a non-treatment portion that is interspersed among the treatment regions.

29. A method of increasing a metabolic rate, the method comprising:
directing energy, using an energy source, at a target region in skin tissue of a subject;
creating a plurality of treatment regions in the target region of the skin tissue, from the energy interacting with the skin tissue at the target region, the plurality of treatment portions being interspersed among an untreated region of the target region of the skin tissue; and
increasing the basal metabolic rate of the subject, from the creation of the plurality of treatment regions.

30. The method of claim 29, wherein each treatment portion has a width that is less than or equal to 1 millimeter.

31. The method of claim 29, further comprising at least one of:
decreasing an amount of fat at the target region of the skin tissue, from the creation of the plurality of treatment regions;
decreasing an amount of fat at a region of the skin tissue that is different than the target region, from the creation of the plurality of treatment regions;
decreasing a thickness of fat at the target region of the skin tissue, from the creation of the plurality of treatment regions;
decreasing a thickness of fat at the region of the skin tissue that is different than the target region, from the creation of the plurality of treatment regions; or
decreasing a total amount of fat of the subject, from the creation of the plurality of treatment regions.

32. The method of claim 29, further comprising converting at least one white fat cell into a beige or a brown fat cell, from the creation of the plurality of treatment regions.

33. The method of claim 29, further comprising:
increasing the concentration of at least one hormone circulating in the subject, from the creation of the plurality of treatment regions; or
increasing the concentration of at least one neurotransmitter circulating in the subject, from the creation of the plurality of treatment regions.

34. The method of claim 33, wherein the at least one hormone or the at least one neurotransmitter is norepinephrine.

35. The method of claim 29, wherein the plurality of treatment regions are created without puncturing or incising the skin tissue.

36. The method of claim 29, further comprising:
moving the energy source to the target region; and
with the energy source stationary, directing energy at the target region from the energy source to create the plurality of treatment regions.

37. The method of claim 35, further comprising:
with the energy source stationary, directing first energy at the target region from the energy source to create a first subset of the plurality of treatment regions; and
with the energy source stationary, directing second energy at the target region from the energy source to create a second subset of the plurality of treatment regions.

38. The method of claim 37, wherein the first subset of treatment regions is a first row of treatment regions, and the second subset of the treatment regions is a second row of treatment regions.

39. The method of claim 37, wherein the first subset of treatment regions is a first column of treatment regions, and the second subset of the treatment regions is a second column of treatment regions.

40. The method of claim 35, wherein the target region is a first target region and further comprising:
moving the energy source to second target region that is different than the first target region; and
with the energy source stationary, directing energy at the second target region from the energy source to create another plurality of treatment regions within the second target region, the another plurality of treatment regions being interspersed with a plurality of non-treatment regions within the second target region of the skin tissue.

41. A method of improving a weight disorder, the method comprising:
directing energy, using an energy source, at a target region in skin tissue of a subject;
creating a plurality of treatment regions in the target region of the skin tissue, from the energy interacting with the skin tissue at the target region, the plurality of treatment regions being interspersed among an untreated region of the target region of the skin tissue;
increasing the basal metabolic rate the subject, from the creation of the plurality of treatment regions;
decreasing an amount of fat of the subject, based on the increasing the basal metabolic rate of the subject; and
improving the weight disorder from the decreasing of the amount of fat of the subject.

42. The method of claim 41, wherein each treatment region has a width that is less than or equal to 1 millimeter.

43. The method of claim 41, further comprising at least one of:
decreasing the amount of fat at the target region of the skin tissue, from the creation of the plurality of treatment regions;
decreasing the amount of fat at a region of the skin tissue that is different than the target region, from the creation of the plurality of treatment regions;
decreasing a thickness of fat at the target region of the skin tissue, from the creation of the plurality of treatment regions;
decreasing a thickness of fat at the region of the skin tissue that is different than the target region, from the creation of the plurality of treatment regions; or
decreasing a total amount of fat of the subject, from the creation of the plurality of treatment regions.

44. The method of claim 41, further comprising converting at least one white fat cell into a beige or a brown fat cell, from the creation of the plurality of treatment regions.

45. The method of claim 41, further comprising:
increasing the concentration of at least one hormone circulating in the subject, from the creation of the plurality of treatment regions; or
increasing the concentration of at least one neurotransmitter circulating in the subject, from the creation of the plurality of treatment regions.

46. The method of claim 45, wherein the at least one hormone or the at least one neurotransmitter is norepinephrine.

47. The method of claim 41, wherein the plurality of treatment regions are created without puncturing or incising the skin tissue.

48. The method of claim 41, further comprising improving one or more diseases caused by the weight disorder, from the creation of the plurality of treatment regions.

49. The method of claim 48, wherein the one or more diseases include at least one of diabetes, heart disease, high blood pressure, mental illness, pain, high cholesterol, or high triglyceride levels.

50. A method of improving one or more diseases, the method comprising:
directing energy, using an energy source, at a target region in skin tissue of a subject;
creating a plurality of treatment regions in the target region of the skin tissue, from the energy interacting with the skin tissue at the target region, the plurality of treatment regions being interspersed among a non-treatment region of the target region of the skin tissue;
decreasing an amount of fat of the subject, based on the creation of the plurality of treatment regions; and
improving the one or more diseases, based on the decreasing the amount of fat of the subject.

51. The method of claim 50, wherein the one or more diseases include at least one of diabetes, heart disease, high blood pressure, mental illness, pain, high cholesterol, or high triglyceride levels.

52. A treatment system comprising:
an energy source configured to thermally damage skin tissue of a subject;
a user input device configured to receive a user input;
a computing device configured to:
receive, from the user input device, the user input indicative of one or more operational parameters of the energy source;
based on the user input, control the energy source according to the one or more operational parameters to cause the energy source to thermally damage the skin tissue by creating a plurality of treatment regions within a target region of the skin tissue to increase the amount of noradrenaline circulating through the bloodstream of the subject, wherein the target region includes a non-treatment portion that is interspersed among the treatment regions.
